(19) United States
(12) Patent Application Publication
(10) Pub. No.: US 2010/0280061 A1
(43) Pub. Date: Nov. 4, 2010

(54) NOVEL ANALGESIC THAT BINDS FILAMIN A

(76) Inventors: Lindsay Burns Barbier, Palo Alto, CA (US); Hoau-Yan Wang, Philadelphia, PA (US); Nan-Horng Lin, Vernon Hills, IL (US); Andrei Blasko, San Bruno, CA (US)

Correspondence Address: Husch Blackwell Sanders, LLP;Husch Blackwell Sanders LLP Welsh & Katz, 120 S RIVERSIDE PLAZA, 22ND FLOOR, CHICAGO, IL, 60606, US

(21) Appl. No.: 12/435,355

(22) Filed: May 4, 2009

(51) Int. Cl. A61K 31/4355; A61P 29/00

(52) U.S. Cl. 514/291

(57) ABSTRACT

A compound, composition and method are disclosed that can bind to FLNA and provide analgesia. A contemplated compound has a structure that corresponds to Formula I, wherein R¹ and R² are substituents on W that is a ring structure, R³ and R⁴ are substituents on the depicted nitrogen atom, m, n and the dotted lines are all defined within.

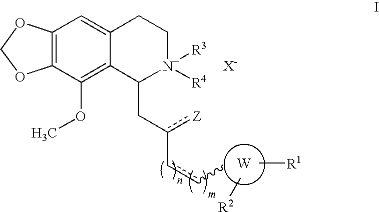

### CROSS-REFERENCE TO RELATED APPLICATION

This applications claims priority from application Ser. No. 12/263,257 that was filed on Oct. 31, 2008, and whose disclosures are incorporated herein by reference.

### TECHNICAL FIELD

This invention contemplates a composition and related method for providing opioid-strength analgesia while minimizing analgesic tolerance, physical dependence and addiction. More particularly, a composition and method are described that utilize a small molecule to inhibit the interaction of the mu opioid receptor with filamin A, either by binding to filamin A itself or by mimicking filamin A's binding to the mu opioid receptor. Preferably, the composition prevents this mu opioid receptor—filamin A interaction and also functions as a mu opioid receptor agonist. Most preferably, the composition binds filamin A with picomolar or sub-picomolar affinity.

### BACKGROUND OF THE INVENTION

Opiates are powerful analgesics (agents used for the treatment of pain), but their use is hampered by non-trivial side effects, tolerance to the analgesic effects, physical dependence resulting in withdrawal effects, and by concerns surrounding the possibility of addiction.

Opiates produce analgesia by activation of opioid receptors that belong to the rhodopsin-like superfamily of G protein-coupled receptors (GPCRs). Adaptive responses of opioid receptors contribute to the development of analgesic tolerance and physical dependence, and possibly also to components of opioid addiction.

Opiates produce analgesia by activation of mu (μ) opioid receptor-linked inhibitory G protein signaling cascades and related ion channel interactions that suppress cellular activities by hyperpolarization. The μ opioid receptor (MOR) preferentially couples to pertussis toxin-sensitive G proteins, Gαi/o (inhibitory/other), and inhibits the adenylyl cyclase/cAMP pathway (Laugwitz et al., 1993 Neuron 10:233-242; Connor et al., 1999 Clin Exp Pharmacol Physiol 26:493-499). The analgesic effects of MOR activation have been predominantly attributed to the Gβγ dimer released from the Gαi/o protein, which activates G protein activated inwardly rectifying potassium (GIRK) channels (Ikeda et al., 2000 Neurosci Res 38:113-116) and inhibits voltage-dependent calcium channels (VDCCs) (Saegusa et al., 2000 Proc Natl Acad Sci USA 97:6132-6137), thereby suppressing cellular activities by hyperpolarization.

Adenylyl cyclase inhibition can also contribute to opioid analgesia, or importantly, its activation can contribute to analgesic tolerance. The role of adenylyl cyclase inhibition or activation in opioid analgesia and analgesic tolerance, respectively, is evidenced by overexpression of adenylyl cyclase type 7 in the CNS of mice leading to more rapid tolerance to morphine (Yoshimura et al., 2000 Mol Pharmacol 58:1011-1016). Additionally, adenylyl cyclase activation has been suggested to elicit analgesic tolerance or tolerance-associated hyperalgesia (Wang et al., 1997 J Neurochem 68:248-254). Although the superactivation of adenylyl cyclase after chronic opioid administration is more often viewed as a hallmark of opioid dependence than as a mediator of tolerance (Nestler, 2001 Am J Addict 10:201-217), both are consequences of chronic opioid administration, and tolerance often worsens dependence. Chronic pain patients who have escalated their opioid dose over time often experience more withdrawal than patients on a constant dose.

An important but underemphasized cellular consequence of chronic opioid treatment is MOR excitatory signaling, by activation of adenylyl cyclase, in place of the usual inhibitory signaling or inhibition of adenylyl cyclase (Crain et al., 1992 Brain Res 575:13-24; Crain et al., 2000 Pain 84:121-131; Gintzler et al., 2001 Mol Neurobiol 21:21-33; Wang et al., 2005 Neuroscience 135:247-261). This change in signaling is caused by a switch in G protein coupling from Gi/o to Gs (Wang et al., 2005 Neuroscience 135:247-261) and may be a result of the decreased efficiency of coupling to the native G proteins, the usual index of desensitization (Sim et al., 1996 J Neurosci 16:2684-2692) and still commonly considered the reason for analgesic tolerance.

The chronic opioid-induced MOR-G protein coupling switch (Wang et al 2005 Neuroscience 135:247-261; Chakrabarti et al., 2005 Mol Brain Res 135:217-224) is accompanied by stimulation of adenylyl cyclase II and IV by MOR-associated Gβγ dimers (Chakrabarti et al., 1998 Mol Pharmacol 54:655-662; Wang et al., 2005 Neuroscience 135:247-261). The interaction of the Gβγ dimer with adenylyl cyclase had previously been postulated to be the sole signaling change underlying the excitatory effects of opiates (Gintzler et al., 2001 Mol Neurobiol 21:21-33). It has further been shown that the Gβγ that interacts with adenylyl cyclases originates from the Gs protein coupling to MOR and not from the Gi/o proteins native to MOR (Wang et al., 2006 J Neurobiol 66:1302-1310).

Thus, MORs are normally inhibitory G protein-coupled receptors that couple to Gi or Go proteins to inhibit adenylyl cyclase and decrease production of the second messenger cAMP, as well as to suppress cellular activities via ion channel-mediated hyperpolarization. Opioid analgesic tolerance and dependence are also associated with that switch in G protein coupling by MOR from Gi/o to Gs (Wang et al., 2005 Neuroscience 135:247-261). This switch results in activation of adenylyl cyclase that provides essentially opposite, stimulatory, effects on the cell.

Controlling this switch in G protein coupling by MOR is the scaffolding protein filamin A, and compounds that bind a particular segment of filamin A with high affinity, like naloxone (NLX) and naltrexone (NTX), can prevent this switch (Wang et al, 2008 PLoS One 3:e1554) and the associated analgesic tolerance and dependence (Wang et al., 2005 Neuroscience 135:247-261). This switch in G protein coupling also occurs acutely, though transiently, and is potentially linked to the acute rewarding or addictive effects of opioid drugs, through CREB activation as a result of increased cAMP accumulation (Wang et al., 2009 PLoS ONE 4(1):e4282).

Ultra-low-dose NLX or NTX have been shown to enhance opioid analgesia, minimize opioid tolerance and dependence (Crain et al., 1995 Proc Natl Acad Sci USA 92:10540-10544; Powell et al. 2002. JPET 300:588-596), as well as to attenuate the addictive properties of opioids (Leri et al., 2005 Pharmacol Biochem Behav 82:252-262; Olmstead et al., 2005 Psychopharmacology 181:576-581). An ultra-low dose of opioid antagonist was an amount initially based on in vitro studies of nociceptive dorsal root ganglion neurons and on in vivo mouse studies, wherein the amount of the excitatory opioid receptor antagonist administered is about 1000- to about 10,000,000-fold less, preferably about 10,000- to about 1,000,000-fold less than the amount of opioid agonist administered. It has long been hypothesized that ultra-low-dose opioid antagonists enhance analgesia and alleviate tolerance/dependence by blocking the excitatory signaling opioid receptors that underlie opioid tolerance and hyperalgesia (Crain et al., 2000 Pain 84:121-131). Later research has shown that the attenuation of opioid analgesic tolerance, dependence and addictive properties by ultra-low-dose, defined herein, naloxone or naltrexone, occurs by preventing the MOR-Gs coupling that results from chronic opiate administration (Wang et al., 2005 Neuroscience 135:247-261), and that the prevention of MOR-Gs coupling is a result of NLX or NTX binding to filamin A at approximately 4 picomolar affinity (Wang et al, 2008 PLoS One 3:e1554).

Found in all cells of the brain, CREB is a transcription factor implicated in addiction as well as learning and memory and several other experience-dependent, adaptive (or maladaptive) behaviors (Carlezon et al., 2005 Trends Neurosci 28:436-445). In general, CREB is inhibited by acute opioid treatment, an effect that is completely attenuated by chronic opioid treatment, and activated during opioid withdrawal (Guitart et al., 1992 J Neurochem 58:1168-1171). However, a regional mapping study showed that opioid withdrawal activates CREB in locus coeruleus, nucleus accumbens and amygdala but inhibits CREB in lateral ventral tegemental area and dorsal raphe nucleus (Shaw-Luthman et al., 2002 J Neurosci 22:3663-3672).

In the striatum, CREB activation has been viewed as a homeostatic adaptation, attenuating the acute rewarding effects of drugs (Nestler, 2001 Am J Addict 10:201-217; Nestler, 2004 Neuropharmacology 47:24-32). This view is supported by nucleus accumbens overexpression of CREB or a dominant-negative mutant respectively reducing or increasing the rewarding effects of opioids in the conditioned place preference test (Barot et al., 2002 Proc Natl Acad Sci USA 99:11435-11440). In conflict with this view, however, reducing nucleus accumbens CREB via antisense attenuated cocaine reinforcement as assessed in self-administration (Choi et al., 2006 Neuroscience 137:373-383). Clearly, CREB activation is implicated in addiction, but whether it directly contributes to the acute rewarding effects of drugs or initiates a homeostatic regulation thereof appears less clear.

The several-fold increase in pS¹³³CREB reported by Wang et al., 2009 PLoS ONE 4(1):e4282 following acute, high-dose morphine may indicate acute dependence rather than acute rewarding effects. However, the transient nature of the MOR-Gs coupling correlating with this CREB activation suggests otherwise. In fact, the correlation of pS¹³³CREB with the Gs coupling by MOR following this acute high-dose morphine exposure, as well as the similar treatment effects on both, suggest that this alternative signaling mode of MOR can contribute to the acute rewarding or addictive effects of opioids. This counterintuitive notion can explain the apparent paradox that ultra-low-dose NTX, while enhancing the analgesic effects of opioids, decreases the acute rewarding or addictive properties of morphine or oxycodone as measured in conditioned place preference or self-administration and reinstatement paradigms.

In considering analgesic tolerance, opioid dependence, and opioid addiction together as adaptive regulations to continued opioid exposure, a treatment that prevents MOR's signaling adaptation of switching its G protein partner can logically attenuate these seemingly divergent behavioral consequences of chronic opioid exposure. However, the acute rewarding effects of opioids are not completely blocked by ultra-low-dose opioid antagonists, suggesting that a MOR-Gs coupling can only partially contribute to the addictive or rewarding effects.

Even though ultra-low-dose NTX blocks the conditioned place preference to oxycodone or morphine (Olmstead et al., 2005 Psychopharmacology 181:576-581), its co-self-administration only reduces the rewarding potency of these opioids but does not abolish self-administration outright (Leri et al., 2005 Pharmacol Biochem Behav 82:252-262). Nevertheless, it is possible that a direct stimulatory effect on VTA neurons, as opposed to the proposed disinhibition via inhibition of GABA interneurons (Spanagel et al., 1993 Proc Natl Acad Sci USA 89:2046-2050), can play some role in opioid reward. Finally, a MOR-Gs coupling mediation of reward, increasing with increasing drug exposure, is in keeping with current theories that the escalation of drug use signifying drug dependence can not indicate a “tolerance” to rewarding effects but instead a sensitization to rewarding effects (Zernig et al., 2007 Pharmacology 80:65-119).

The above results reported in Wang et al., 2009 PLoS ONE 4(1):e4282 demonstrated that acute, high-dose morphine causes an immediate but transient switch in G protein coupling by MOR from Go to Gs similar to the persistent switch caused by chronic morphine. Ultra-low-dose NLX or NTX prevented this switch and attenuated the chronic morphine-induced coupling switch by MOR. The transient nature of this acute altered coupling suggests the receptor eventually recovers and couples to its native G protein.

With chronic opioid exposure, the receptor can lose the ability to recover and continue to couple to Gs, activating the adenylyl cyclase/cAMP pathway, upregulating protein kinase A, and phosphorylating CREB as one downstream effector example. The persistently elevated phosphorylated CREB can then shape the expression of responsive genes including those closely related to drug addiction and tolerance. Importantly, the equivalent blockade of Gs coupling and pS¹³³CREB by the pentapeptide binding site of naloxone (NLX) and naltrexone (NTX) on FLNA further elucidates the mechanism of action of ultra-low-dose NLX and NTX in their varied effects.

These data further strengthen a mechanistic basis for MOR-Gs coupling through the interaction between FLNA and MOR and that disrupting this interaction, either by NLX/NTX binding to FLNA or via a FLNA peptide decoy for MOR, the altered coupling is prevented, resulting in attenuation of tolerance, dependence and addictive properties associated with opioid drugs.

The combination of ultra-low-dose opioid antagonists with opioid agonists formulated together in one medication has been shown to alleviate many of these undesirable aspects of opioid therapy (Burns, 2005 Recent Developments in Pain Research 115-136, ISBN:81-308-0012-8). This approach shows promise for an improvement in analgesic efficacy, and animal data suggests reduced addictive potential. The identification of the cellular target of ultra-low-dose NLX or NTX in their inhibition of mu opioid receptor-Gs coupling as a pentapeptide segment of filamin A (Wang et al., 2008 PLoS ONE 3(2):e1554) has led to development of assays to screen against this target to create a new generation of pain therapeutics that can provide long-lasting analgesia with minimal tolerance, dependence and addictive properties. Importantly, the non-opioid cellular target of ultra-low-dose NLX or NTX, FLNA, provides potential for developing either a therapeutic combination of which one component is not required to be ultra-low-dose, or a single-entity novel analgesic.

The present invention identifies a compound that is similar to or more active than DAMGO in activating the mu (μ) opioid receptor (MOR), and that also binds to filamin A (FLNA; the high-affinity binding site of naloxone [NLX] and naltrexone [NTX]), thereby preventing the Gi/o-to-Gs coupling switch of MOR to attenuate opioid tolerance, dependence and addiction.

### BRIEF SUMMARY OF THE INVENTION

The present invention contemplates a method of reducing pain in a host mammal in need thereof by administering administering to a host mammal in need thereof a pharmaceutical composition containing an analgesia effective amount of a compound of Formula I dissolved or dispersed in a physiologically tolerable carrier.

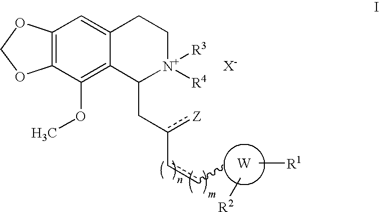

wherein

n=0 or 1;

m=0 or 1;

m+n=0, 1 or 2;

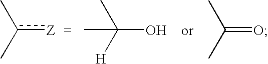

W is an aromatic ring containing 0, 1 or 2 hetero atoms that can be nitrogen, oxygen or sulfur, or mixtures thereof in the ring;

R¹ is selected from the group consisting of H, C₁-C₆ hydrocarbyl, C₁-C₆ hydrocarbyloxy, halogen, cyano, C₁-C₆ hydrocarbyloxyhydrocarboxylene, trifluoromethyl, and hydroxyl;

R² is selected from the group consisting of H, C₁-C₆ hydrocarbyl, C₁-C₆ hydrocarboylxy, C₁-C₆ hydrocarbyloxyhydrocarboxylene and halogen;

R³ is absent or C₁-C₆ hydrocarbyl;

R⁴ is C₁-C₆ hydrocarbyl;

X⁻=an anion or is absent when R³ is absent;

the dotted line indicates an optional double bond between the depicted carbon atoms; and

the wavy line indicates that the depicted phenyl substituent can be in the Z or E configuration when the optional double bond is present.

In preferred practice, the sum of m+n is 1 or 2. In some embodiments, W contains zero nitrogen atoms, whereas in other embodiments, W contains one nitrogen atom. In some preferred embodiments, R²═H. It is also sometimes preferred that R¹ include an oxygen atom bonded to W, as where R¹ is C₁-C₆ hydrocarbyloxy. It is also preferred in some embodiments that

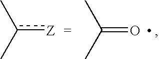

whereas in other embodiments it is preferred that

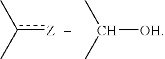

In some embodiments, R³═CH₃, whereas in other embodiments, R³═H.

A contemplated composition is typically administered a plurality of times over a period of days, and is preferably administered a plurality of times in one day.

The present invention has several benefits and advantages.

One benefit is that analgesia can be provided at morphine-like potency by a compound that does not have a narcotic structure.

An advantage of the invention is that analgesia can be provided by administration of a contemplated composition either perorally or parenterally.

A further benefit of the invention is that as indicated by the initial data, a contemplated compound provides the analgesic effects characteristic of opioid drugs but does not cause analgesic tolerance or dependence.

Another advantage of the invention as also indicated by the initial data is that a contemplated compound provides the analgesic effects and does not have the addictive potential of opioid drugs.

Still further benefits and advantages will be apparent to a skilled worker from the description that follows.

### ABBREVIATIONS AND SHORT FORMS

The following abbreviations and short forms are used in this specification.

“MOR” means μ-opioid receptor

“FLNA” means filamin A

“NLX” means naloxone

“NTX” means naltrexone

“Gαi/o” means G protein alpha subunit—inhibitory/other conformation, inhibits adenylyl cyclase

“Gαs” means G protein alpha subunit—stimulatory conformation stimulates adenylyl cyclase

“Gβγ” means G protein beta gamma subunit

“cAMP” means cyclic adenosine monophosphate

“CREB” means cAMP Response Element Binding protein

“IgG” means Immunoglobulin G

### DEFINITIONS

In the context of the present invention and the associated claims, the following terms have the following meanings:

The articles “a” and “an” are used herein to refer to one or to more than one (i.e., to at least one) of the grammatical object of the article. By way of example, “an element” means one element or more than one element.

As used herein, the term “hydrocarbyl” is a short hand term to include straight and branched chain aliphatic as well as alicyclic groups or radicals that contain only carbon and hydrogen. Thus, alkyl, alkenyl and alkynyl groups are contemplated, whereas aromatic hydrocarbons such as phenyl and naphthyl groups, which strictly speaking are also hydrocarbyl groups, are referred to herein as aryl groups, substituents, moieties or radicals, as discussed hereinafter. An aralkyl group such as benzyl or phenethyl is deemed a hydrocarbyl group. Where a specific aliphatic hydrocarbyl substituent group is intended, that group is recited; i.e., C₁-C₄ alkyl, methyl or dodecenyl. Exemplary hydrocarbyl groups contain a chain of 1 to about 12 carbon atoms, and preferably one to about 7 carbon atoms, and preferably 1 to about 7 carbon atoms, and more preferably 1 to 4 carbon atoms of an alkyl group.

A particularly preferred hydrocarbyl group is an alkyl group. As a consequence, a generalized, but more preferred substituent can be recited by replacing the descriptor “hydrocarbyl” with “alkyl” in any of the substituent groups enumerated herein.

Examples of alkyl radicals include methyl, ethyl, n-propyl, isopropyl, n-butyl, isobutyl, sec-butyl, tert-butyl, pentyl, iso-amyl, hexyl, octyl, decyl, dodecyl and the like. Examples of suitable alkenyl radicals include ethenyl(vinyl), 2-propenyl, 3-propenyl, 1,4-pentadienyl, 1,4-butadienyl, 1-butenyl, 2-butenyl, 3-butenyl, decenyl and the like. Examples of alkynyl radicals include ethynyl, 2-propynyl, 3-propynyl, decynyl, 1-butynyl, 2-butynyl, 3-butynyl, and the like.

Usual chemical suffix nomenclature is followed when using the word “hydrocarbyl” except that the usual practice of removing the terminal “yl” and adding an appropriate suffix is not always followed because of the possible similarity of a resulting name to one or more substituents. Thus, a hydrocarbyl ether is referred to as a “hydrocarbyloxy” group rather than a “hydrocarboxy” group as may possibly be more proper when following the usual rules of chemical nomenclature. Illustrative hydrocarbyloxy groups include methoxy, ethoxy, and cyclohexenyloxy groups. On the other hand, a hydrocarbyl group containing a —C(O)o— functionalityis referred to as a hydrocarboyl(acyl) or hydrocarboyloxy group inasmuch as there is no ambiguity. Exemplary hydrocarboyl and hydrocarboyloxy groups include acyl and acyloxy groups, respectively, such as acetyl and acetoxy, acryloyl and acryloyloxy.

A “carboxyl” substituent is a —C(O)OH group. A C₁-C₆ hydrocarbyl carboxylate is a C₁-C₆ hydrocarbyl ester of a carboxyl group. A carboxamide is a —C(O)NR₃R₄ substituent, where the R groups are defined elsewhere. Illustrative R³ and R⁴ groups that together with the depicted nitrogen of a carboxamide form a 5-7-membered ring that optionally contains 1 or 2 additional hetero atoms that independently are nitrogen, oxygen or sulfur, include morpholinyl, piprazinyl, oxathiazolyl, 1,2,3-triazolyl, 1,2,4-triazolyl, pyrazolyl, 1,2,4-oxadiazinyl and azepinyl groups.

As a skilled worker will understand, a substituent that cannot exist such as a C₁ alkenyl or alkynyl group is not intended to be encompassed by the word “hydrocarbyl”, although such substituents with two or more carbon atoms are intended.

The term “aryl”, alone or in combination, means a phenyl or naphthyl radical that optionally carries one or more substituents selected from hydrocarbyl, hydrocarbyloxy, halogen, hydroxy, amino, nitro and the like, such as phenyl, p-tolyl, 4-methoxyphenyl, 4-(tert-butoxy)phenyl, 4-fluorophenyl, 4-chlorophenyl, 4-hydroxyphenyl, and the like. The term “arylhydrocarbyl”, alone or in combination, means a hydrocarbyl radical as defined above in which one hydrogen atom is replaced by an aryl radical as defined above, such as benzyl, 2-phenylethyl and the like. The term “arylhydrocarbyloxycarbonyl”, alone or in combination, means a radical of the formula —C(O)—O-arylhydrocarbyl in which the term “arylhydrocarbyl” has the significance given above. An example of an arylhydrocarbyloxycarbonyl radical is benzyloxycarbonyl. The term “aryloxy” means a radical of the formula aryl-O— in which the term aryl has the significance given above. The term “aromatic ring” in combinations such as substituted-aromatic ring sulfonamide, substituted-aromatic ring sulfinamide or substituted-aromatic ring sulfenamide means aryl or heteroaryl as defined above.

As used herein, the term “binds” refers to the adherence of molecules to one another, such as, but not limited to, peptides or small molecules such as the compounds disclosed herein, and opioid antagonists, such as naloxone or naltrexone.

As used herein, the term “selectively binds” refers to binding as a distinct activity. Examples of such distinct activities include the independent binding to filamin A or a filamin A binding peptide, and the binding of a compound discussed above to a p opioid receptor.

As used herein, the term “FLNA-binding compound” refers to a compound that binds to the scaffolding protein filamin A, or more preferably to a polypeptide comprising residues -Val-Ala-Lys-Gly-Leu- (SEQ ID NO:1) of the FLNA sequence that correspond to amino acid residue positions 2561-2565 of the FLNA protein sequence as noted in the sequence provided at the web address: UniProtKB/Swiss-Prot entry P21333, FLNA-HUMAN, Filamin-A protein sequence. A FLNA-binding compound can inhibit the MOR-Gs coupling caused by agonist stimulation of the p opioid receptor via interactions with filamin A, preferably in the 24^{th} repeat region. When co-administered with an opioid agonist, a FLNA-binding compound can enhance the analgesic effects and improve the treatment of pain.

As used herein, the term “candidate FLNA-binding compound” refers to a substance to be screened as a potential FLNA-binding compound. In preferred instances a FLNA-binding compound is also an opioid agonist. Additionally, a FLNA-binding compound can function in a combinatory manner similar to the combination of an opioid agonist and ultra-low-dose antagonist, wherein both FLNA and the mu-opioid receptor are targeted by a single entity.

As used herein, the term “opioid receptor” refers to a G protein coupled receptor, located in the central nervous system that interacts with opioids. More specifically, the p opioid receptor is activated by morphine causing analgesia, sedation, nausea, and many other side effects known to one of ordinary skill in the art.

As used herein, the term “opioid agonist” refers to a substance that upon binding to an opioid receptor can stimulate the receptor, induce G protein coupling and trigger a physiological response. More specifically, an opioid agonist is a morphine-like substance that interacts with MOR to produce analgesia.

As used herein, the term “opioid antagonist” refers to a substance that upon binding to an opioid receptor inhibits the function of an opioid agonist by interfering with the binding of the opioid agonist to the receptor.

As used herein an “analgesia effective amount” refers to an amount sufficient to provide analgesia or pain reduction to a recipient host.

As used herein the term “ultra-low-dose” or “ultra-low amount” refers to an amount of compound that when given in combination with an opioid agonist is sufficient to enhance the analgesic potency of the opioid agonist. More specifically, the ultra-low-dose of an opioid antagonist admixed with an opioid agonist in mammalian cells is an amount about 1000- to about 10,000,000-fold less, and preferably between about 10,000- and to about 1,000,000-fold less than the amount of opioid agonist.

As used herein an “FLNA-binding effective amount” refers to an amount sufficient to perform the functions described herein, such as inhibition of MOR-Gs coupling, prevention of the cAMP desensitization measure, inhibition of CREB S¹³³ phosphorylation and inhibition of any other cellular indices of opioid tolerance and dependence, which functions can also be ascribed to ultra-low-doses of certain opioid antagonists such as naloxone or naltrexone. When a polypeptide or FLNA-binding compound of the invention interacts with FLNA, an FLNA-binding effective amount can be an ultra-low amount or an amount higher than an ultra-low-dose as the polypeptide or FLNA-binding compound will not antagonize the opioid receptor and compete with the agonist, as occurs with known opioid antagonists such as naloxone or naltrexone in amounts greater than ultra-low-doses. More preferably, when a polypeptide or VAKGL-binding compound of the present invention both interacts with FLNA and is an agonist of the mu opioid receptor, an FLNA-binding effective amount is an amount higher than an ultra-low-dose and is a sufficient amount to activate the mu opioid receptor.

As used herein the phrase “determining inhibition of the interaction of a mu opioid receptor with a Gs protein” refers to monitoring the cellular index of opioid tolerance and dependence caused by chronic or high-dose administration of opioid agonists to mammalian cells. More specifically, the mu opioid receptor—Gs coupling response can be identified by measuring the presence of the Gαs (stimulatory) subunit, the interaction of MOR with the G protein complexes and formation of Gs-MOR coupling, the interaction of the Gpy protein with adenylyl cyclase types II and IV, loss of inhibition or outright enhancement of cAMP accumulation, and the activation of CREB via phosphorylation of S¹³³.

As used herein the term “naloxone/naltrexone positive control” refers to a positive control method comprising steps discussed in a method embodiment, wherein the candidate FLNA-binding compound is a known opioid antagonist administered in an ultra-low amount, preferably naloxone or naltrexone.

As used herein the term “FLNA-binding compound negative control” refers to a negative control method comprising steps discussed in a method embodiment, wherein the candidate FLNA-binding compound is absent and the method is carried out in the presence of only opioid agonist.

As used herein the term “pharmacophore” is not meant to imply any pharmacological activity. The term refers to chemical features and their distribution in three-dimensional space that constitutes and epitomizes the preferred requirements for molecular interaction with a receptor (U.S. Pat. No. 6,034,066).

### DETAILED DESCRIPTION OF THE INVENTION

It should be understood that the present disclosure is to be considered as an exemplification of the present invention, and is not intended to limit the invention to the specific embodiments illustrated. It should be further understood that the title of this section of this application (“Detailed Description of the Invention”) relates to a requirement of the United States Patent Office, and should not be found to limit the subject matter disclosed herein.

The present invention contemplates a compound that binds to FLNA and also stimulates the p opioid receptor (MOR), and method of its use to provide analgesia. A contemplated compound can inhibit MOR-Gs coupling through interactions with FLNA and/or the p opioid receptor (MOR).

In another aspect of the present invention, a contemplated compound prevents the morphine-induced Gs protein coupling by MOR. That prevention of MOR-Gs coupling is believed to occur by preventing the interaction of filamin A and MOR. Downstream effects of preventing the MOR-Gs coupling include inhibition of cAMP accumulation and of cAMP Response Element Binding protein (CREB) activation in a manner resembling the activity of ultra-low-dose opioid antagonists naloxone and naltrexone.

In another aspect of the present invention, a FLNA-binding compound prevents the MOR-Gs coupling while itself activating MOR.

The data collected in organotypic striatal slice cultures demonstrate that after 7 days of twice daily 1-hour exposures to oxycodone, mu opioid receptors in striatum switch from Go to Gs coupling (compare vehicle to oxycodone conditions). In contrast, a compound contemplated herein did not cause a switch to Gs coupling despite its ability to stimulate mu opioid receptors as previously assessed by GTPγS binding that is blocked by beta-funaltrexamine, a specific mu opioid receptor antagonist. These data imply that these novel compounds provide the analgesic effects characteristic of opioid drugs but do not cause analgesic tolerance or dependence, and do not have the addictive potential of opioid drugs.

A compound contemplated by the present invention binds to an above-defined FLNA polypeptide as well as stimulates the μ opioid receptor (MOR). A contemplated compound useful in a method of the invention corresponds in structure to Formula I, below. Many of these compounds have been reported in the literature and can be found, for example, in Eckhart et al. 1962 Periodica Polytech., 6(1):57-64; Eckhart et al., 1961 Magyar Kemiai Folyoirat, 67:509-511; and Kaneko et al., 1964 Yakugaku Zasshi, 84(10):988-992.

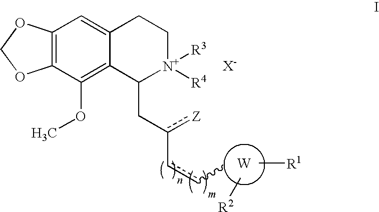

wherein

n=0 or 1;

m=0 or 1;

m+n=0, 1 or 2;

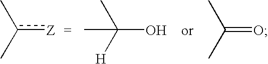

W is an aromatic ring containing 0, 1 or 2 hetero atoms that can be nitrogen, oxygen or sulfur, or mixtures thereof in the ring;

R¹ is selected from the group consisting of H, C₁-C₆ hydrocarbyl, C₁-C₆ hydrocarbyloxy, halogen, cyano, C₁-C₆ hydrocarbyloxyhydrocarboxylene, trifluoromethyl, and hydroxyl;

R² is selected from the group consisting of H, C₁-C₆ hydrocarbyl, C₁-C₆ hydrocarbyloxy, C₁-C₆ hydrocarbyloxyhydrocarboxylene and halogen;

R³ is absent or C₁-C₆ hydrocarbyl;

R⁴ is C₁-C₆ hydrocarbyl;

X⁻=an anion or is absent when R³ is absent;
the dotted line indicates an optional double bond between the depicted carbon atoms; and

the wavy line indicates that the depicted phenyl substituent can be in the Z or E configuration when the optional double bond is present.

Illustrative anions are pharmaceutically acceptable and include sulfate, bisulfate, chloride, bromide, iodide, acetate, formate, benzenesulfonate and the like as are well known. These and other anions are listed in Berge et al., 1977 J. Pharm Sci. 68(1):1-19.

It is preferred that m+n=1 or 2, and the optional double bond is absent and is rather a saturated, single bond.

In preferred practice, W is a six-membered ring, although five membered rings are also contemplated. Thus, a contemplated aromatic ring that can include zero, one or two hetero atoms that are nitrogen, oxygen or sulfur or mixtures thereof include phenyl, pyridyl, furanyl, imidazyl, oxazolyl and the like. In some preferred embodiments, W is free of (has zero) ring nitrogen atoms. In other embodiments, preferred compounds have W groups that are free of ring hetero atoms, having only ring carbon atoms.

W preferably further includes one or more substituent groups (R¹ and R²) to one or more ring atoms, in which those one or more substituents contain a total of up to 12 atoms selected from the group consisting of carbon, nitrogen, oxygen and sulfur, and mixtures thereof. Preferred substituent groups on ring W have an oxygen atom bonded to the W ring. Such compounds are preferably C₁-C₆ hydrocarbyloxy groups such as methoxy groups.

The Z-containing group can be a keto group or can be a reduce hydroxyl group. Both groups are preferred.

In some embodiments, both R³ and R⁴ are C₁-C₆ hydrocarbyl groups that are both methyl. In other embodiments, one is an ethyl group and the other is methyl or absent. Illustrative compounds where R³ is absent are shown below.

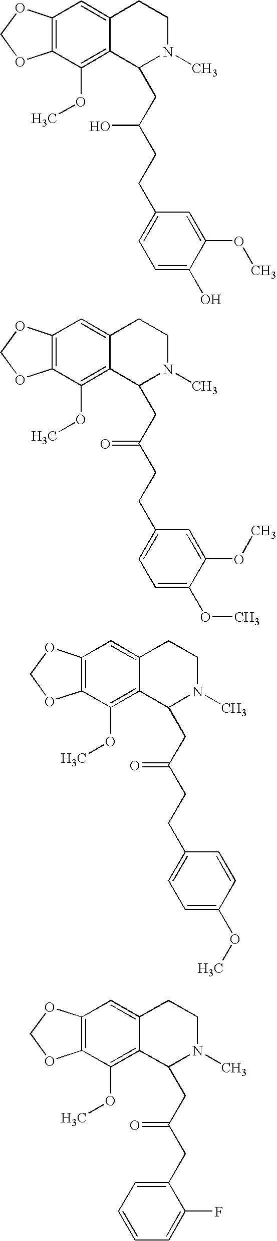

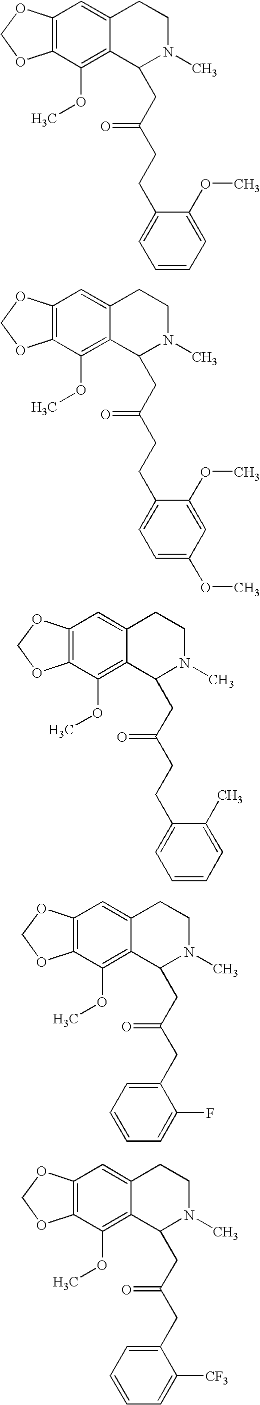

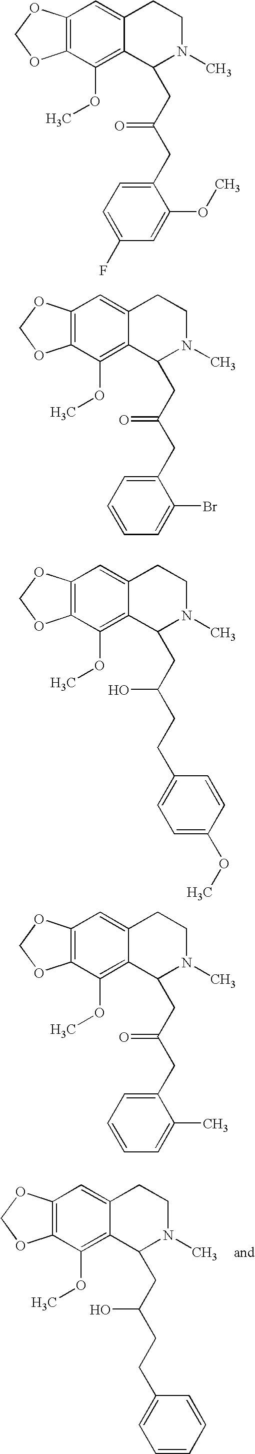

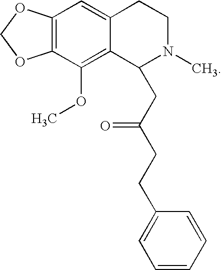

In one preferred embodiment, a compound of Formula I has the structure of Formula II,

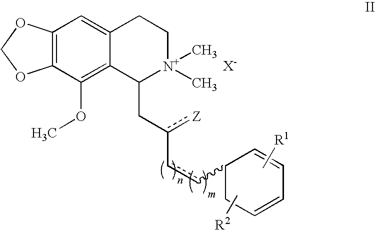

wherein

n=0 or 1;

m=0 or 1;

m+n=0, 1 or 2;

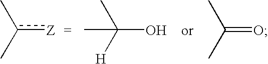

X⁻=an anion;

R¹ is selected from the group consisting of H, C₁-C₆ hydrocarbyl, C₁-C₆ hydrocarbyloxy, halogen, cyano, C₁-C₆ hydrocarbyloxyhydrocarboxylene, trifluoromethyl, and hydroxyl;

R² is selected from the group consisting of H, C₁-C₆ hydrocarbyl, C₁-C₆ hydrocarbyloxy, C₁-C₆ hydrocarbyloxyhydrocarboxylene and halogen;

the dotted line indicates an optional double bond between the depicted carbon atoms; and

the wavy line indicates that the depicted phenyl substituent can be in the Z or E configuration when the optional double bond is present.

In some preferred embodiments, R²═H. In some such embodiments, R¹ includes an oxygen atom bonded to the depicted phenyl ring, and that oxygen is preferably part of a C₁-C₆ hydrocarbyloxy group. For may compounds, it is preferred that

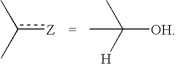

Illustrative preferred compounds of Formula II in clued those shown below.

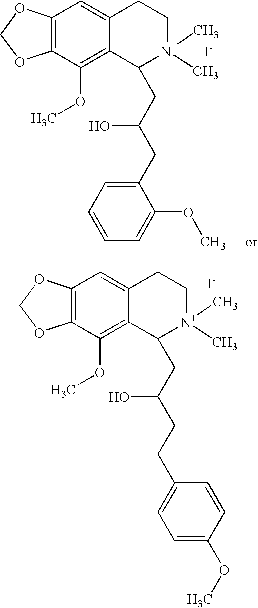

In yet other preferred embodiments, a method of reducing pain in a host mammal in need thereof is contemplated that comprises administering to that host mammal a pharmaceutical composition containing an effective amount of a compound of Formula III dissolved or dispersed in a physiologically tolerable carrier

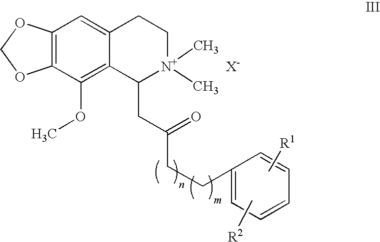

here,

n=0 or 1;

m=0 or 1;

m+n=0, 1 or 2;

X⁻=an anion;

R¹ is selected from the group consisting of H, C₁-C_{G} hydrocarbyl, C₁-C₆ hydrocarbyloxy, halogen, cyano, C₁-C₆ hydrocarbyloxyhydrocarboxylene, trifluoromethyl, and hydroxyl; and

R² is selected from the group consisting of H, C₁-C₆ hydrocarbyl, C₁-C₆ hydrocarbyloxy, C₁-C₆ hydrocarbyloxyhydrocarboxylene and halogen.

As was the case for other embodiments, R² is sometimes H, and one or both of R¹ and R² are C₁-C₆ hydrocarbyloxy groups such as methoxy. A particularly preferred compound of Formula III is selected from the group consisting of

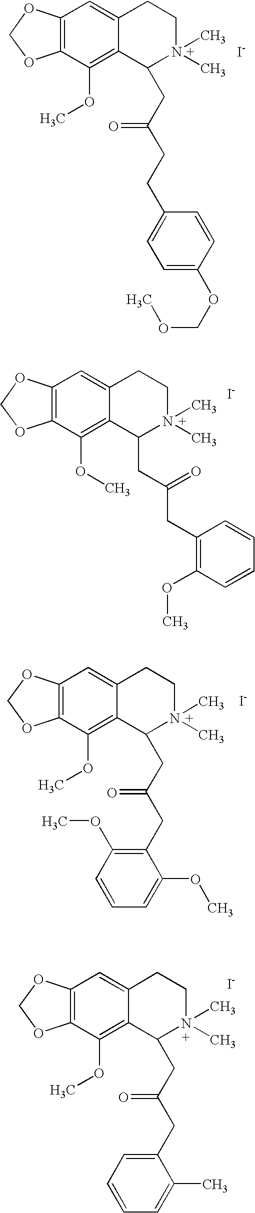

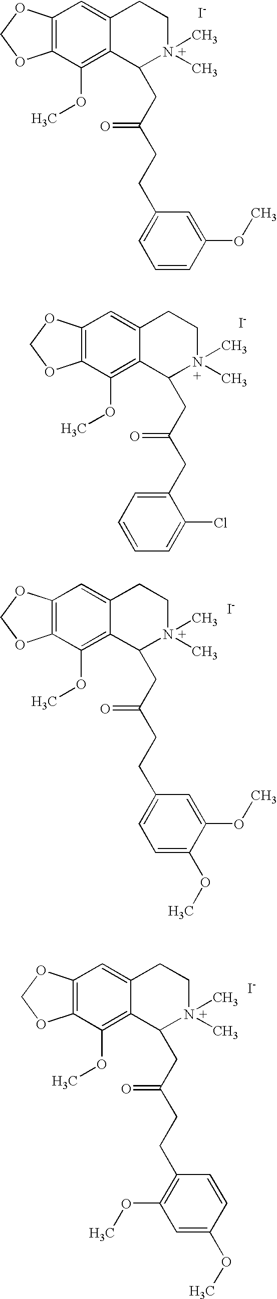

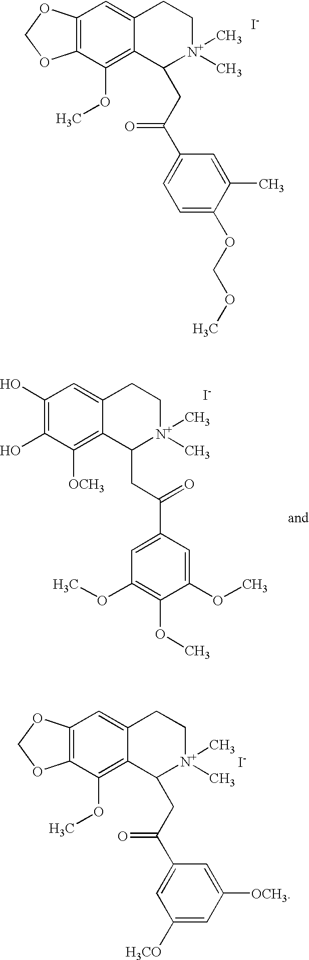

In another aspect, a contemplated compound is selected in part using a method for determining the ability of a candidate FLNA-binding compound, other than naloxone or naltrexone, to inhibit the interaction of the mu opioid receptor with filamin A (FLNA) and thereby prevent the mu opioid receptor from coupling to Gs proteins (Gs). That method comprises the steps of: (a) admixing the candidate FLNA-binding compound (alone if such FLNA-binding compound also stimulates MOR or with a MOR agonist otherwise) with mammalian cells that contain the mu opioid receptor and FLNA in their native conformations and relative orientations, the opioid agonist being present in an agonist effective amount and/or being administered in a repeated, chronic manner the FLNA-binding compound being present in an FLNA-binding effective amount; and (b) determining inhibition of the interaction of the mu opioid receptor with the G protein by analysis of the presence or the absence of the Gαs subunit of Gs protein, wherein the absence of the Gαs subunit indicates inhibition of the interaction of the mu opioid receptor with the Gs protein.

In one aspect, the analysis of Gs protein coupling by the mu opioid receptor and downstream effects elicited by admixing mammalian cells with a beforedefined compound can be conducted by any one or more of several methods such as for example co-immunoprecipitation of Ga proteins with MOR, Western blot detection of MOR in immunoprecipitates, and densitometric quantification of Western blots.

### Pharmaceutical Composition

A pharmaceutical composition is contemplated that contains an analgesia effective amount of a compound of Formula I, Formula II, or Formula III dissolved or dispersed in a physiologically tolerable carrier. Such a composition can be administered to mammalian cells in vitro as in a cell culture, or in vivo as in a living, host mammal in need.

A contemplated composition is typically administered a plurality of times over a period of days. More usually, a contemplated composition is administered a plurality of times in one day.

As is seen from the data that follow, a contemplated compound is active in the assays studies at micromolar amounts. In the laboratory mouse tail flick test, orally administered compound 5009 exhibited a mean maximum antinoniception amount of about 30% at 32 mg/kg at about 20 minutes, whereas compound B0040 exhibited a mean maximum antinoniception amount of about 25% at 32 mg/kg at about 20-30 minutes, and compound B0036 exhibited a mean maximum antinoniception amount of about 20% at 32 mg/kg at about 30 minutes. Morphine administered at the same dose exhibited an antinoniceptive effect of about 30% at thirty minutes. It is thus seen that the contemplated compounds are quite active and potent, and that a skilled worker can readily determine an appropriate dosage level, particularly in view of the relative activity of a contemplated compound compared to orally administered morphine.

A contemplated composition described herein can be used in the manufacture of a medicament that is useful at least for lessening or reducing pain in a mammal that is in need.

A contemplated pharmaceutical composition can be administered orally (perorally), parenterally, by inhalation spray in a formulation containing conventional nontoxic pharmaceutically acceptable carriers, adjuvants, and vehicles as desired. The term parenteral as used herein includes subcutaneous injections, intravenous, intramuscular, intrasternal injection, or infusion techniques. Formulation of drugs is discussed in, for example, Hoover, John E., Remington's Pharmaceutical Sciences, Mack Publishing Co., Easton, Pa.; 1975 and Liberman, H. A. and Lachman, L., Eds., Pharmaceutical Dosage Forms, Marcel Decker, New York, N.Y., 1980.

Injectable preparations, for example, sterile injectable aqueous or oleaginous suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be employed are water, Ringer's solution, and isotonic sodium chloride solution, phosphate-buffered saline. Liquid pharmaceutical compositions include, for example, solutions suitable for parenteral administration. Sterile water solutions of an active component or sterile solution of the active component in solvents comprising water, ethanol, or propylene glycol are examples of liquid compositions suitable for parenteral administration.

In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables. Dimethyl acetamide, surfactants including ionic and non-ionic detergents, polyethylene glycols can be used. Mixtures of solvents and wetting agents such as those discussed above are also useful.

Sterile solutions can be prepared by dissolving the active component in the desired solvent system, and then passing the resulting solution through a membrane filter to sterilize it or, alternatively, by dissolving the sterile compound in a previously sterilized solvent under sterile conditions.

Solid dosage forms for oral administration can include capsules, tablets, pills, powders, and granules. In such solid dosage forms, the compounds of this invention are ordinarily combined with one or more adjuvants appropriate to the indicated route of administration. If administered per os, the compounds can be admixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation as can be provided in a dispersion of active compound in hydroxypropylmethyl cellulose. In the case of capsules, tablets, and pills, the dosage forms can also comprise buffering agents such as sodium citrate, magnesium or calcium carbonate or bicarbonate. Tablets and pills can additionally be prepared with enteric coatings.

A mammal in need of treatment and to which a pharmaceutical composition containing a contemplated compound is administered can be a primate such as a human, an ape such as a chimpanzee or gorilla, a monkey such as a cynomolgus monkey or a macaque, a laboratory animal such as a rat, mouse or rabbit, a companion animal such as a dog, cat, horse, or a food animal such as a cow or steer, sheep, lamb, pig, goat, llama or the like.

Where in vitro mammalian cell contact is contemplated, a CNS tissue culture of cells from an illustrative mammal is often utilized, as is illustrated hereinafter. In addition, a non-CNS tissue preparation that contains opioid receptors such as guinea pig ileumcan also be used.

Preferably, the pharmaceutical composition is in unit dosage form. In such form, the composition is divided into unit doses containing appropriate quantities of the active urea. The unit dosage form can be a packaged preparation, the package containing discrete quantities of the preparation, for example, in vials or ampules.

### EXAMPLES

The present invention is described in the following examples which are set forth to aid in the understanding of the invention, and should not be construed to limit in any way the invention as defined in the claims which follow thereafter.

The experiments described herein were carried out on organotypic striatal slices from male Sprague Dawley rats (200 to 250 g) purchased from Taconic (Germantown, N.Y.). Rats were housed two per cage and maintained on a regular 12-hour light/dark cycle in a climate-controlled room with food and water available ad libitum and sacrificed by rapid decapitation. All data are presented as mean±standard error of the mean. Treatment effects were evaluated by two-way ANOVA followed by Newman-Keul's test for multiple comparisons. Two-tailed Student's t test was used for post hoc pairwise comparisons. The threshold for significance was p<0.05.

The following Table of Correspondence shows the structures of the compounds discussed herein and their identifying numbers.

Table of Correspondence

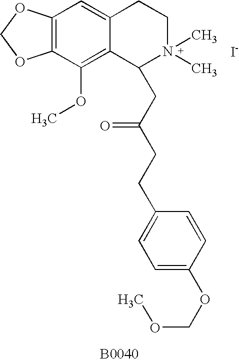

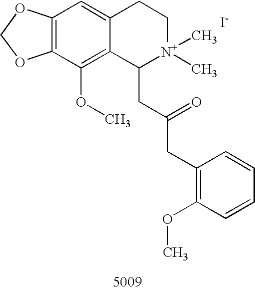

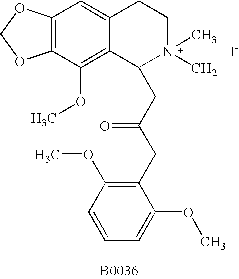

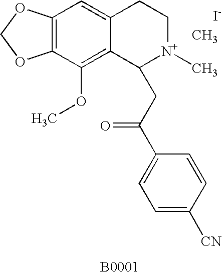

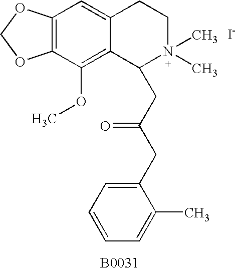

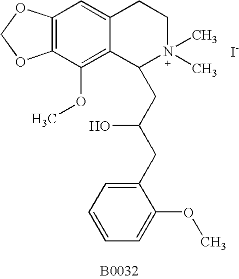

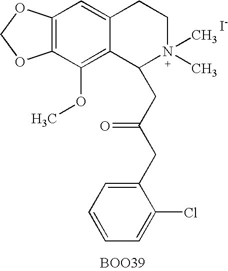

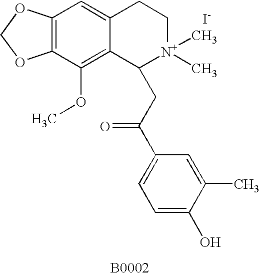

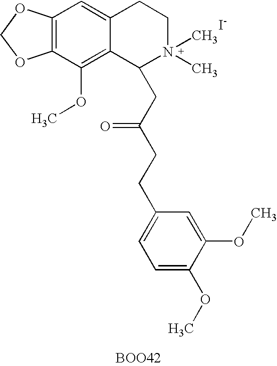

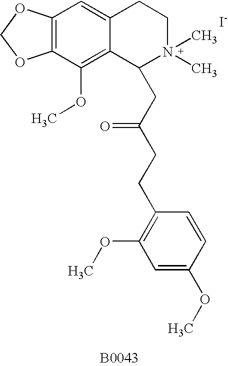

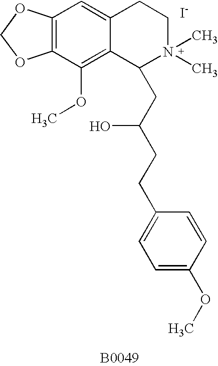

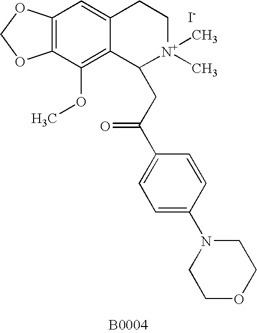

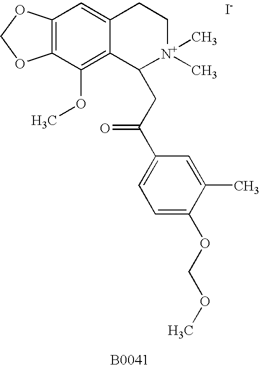

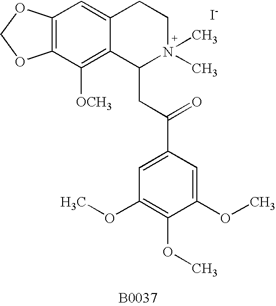

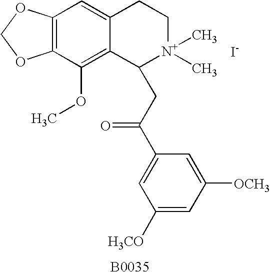

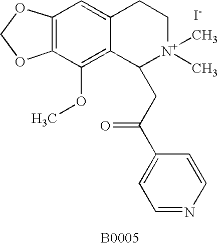

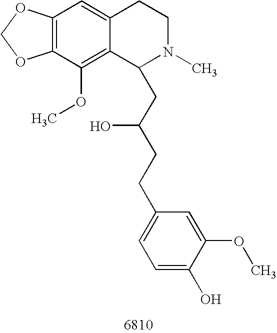

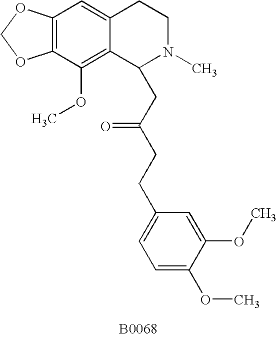

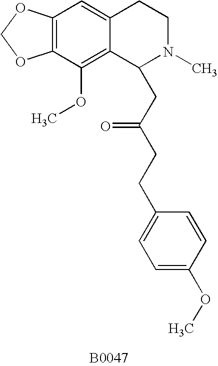

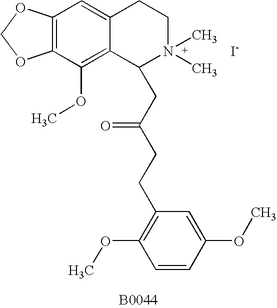

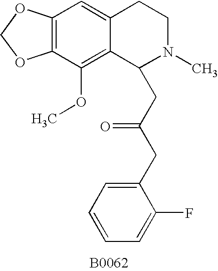

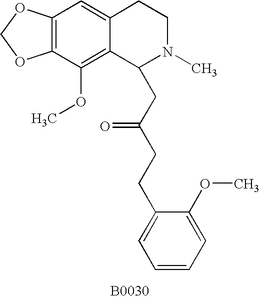

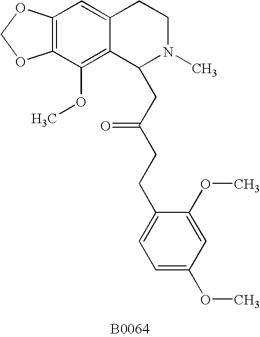

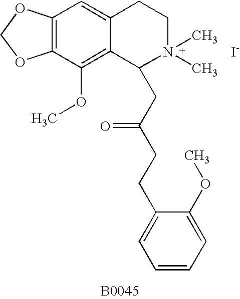

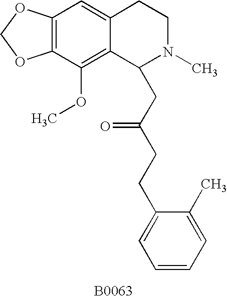

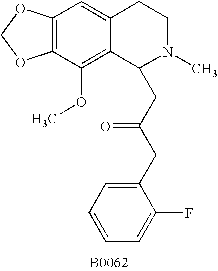

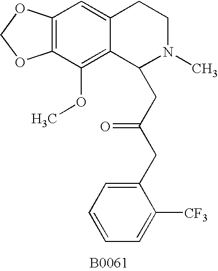

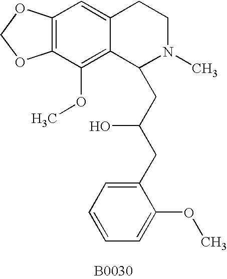

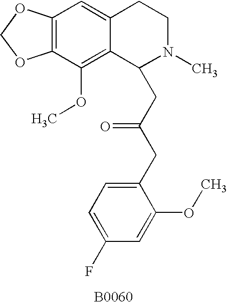

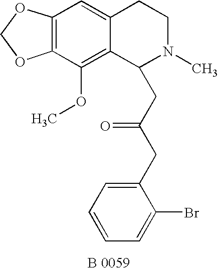

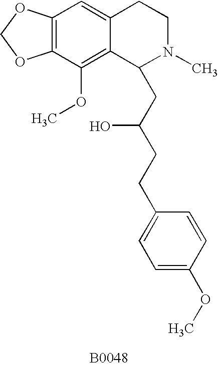

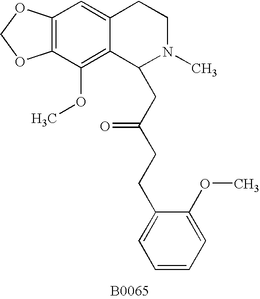

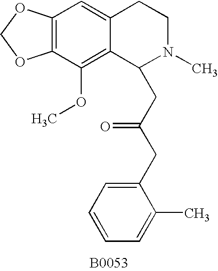

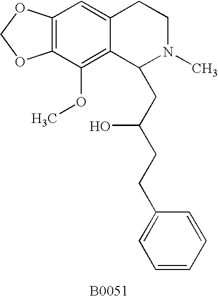

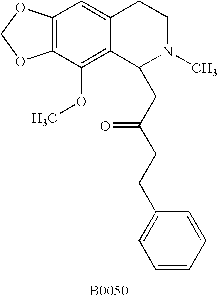

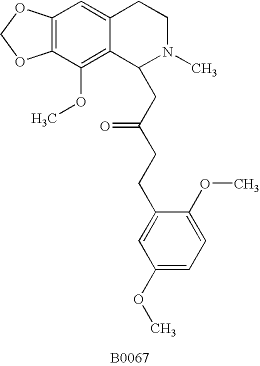

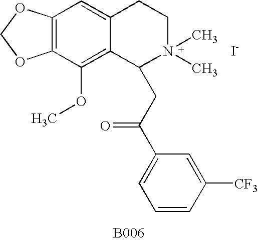

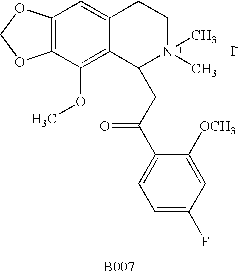

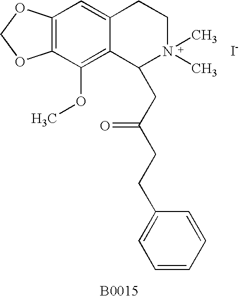

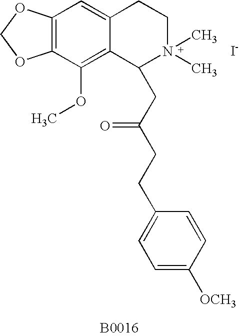

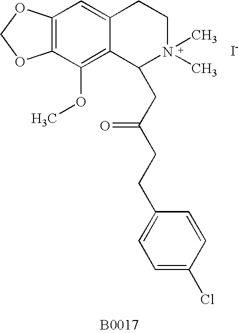

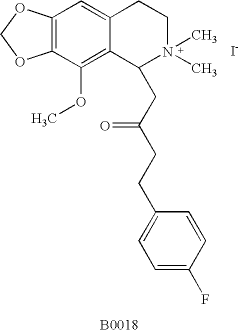

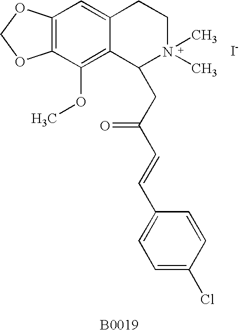

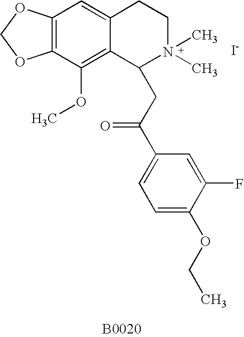

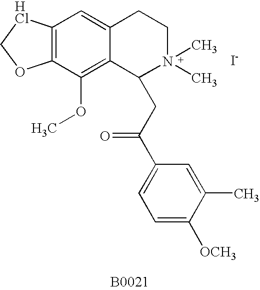

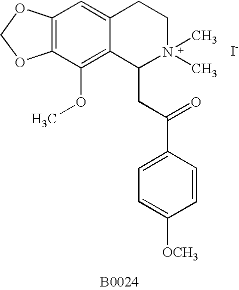

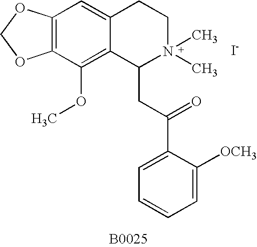

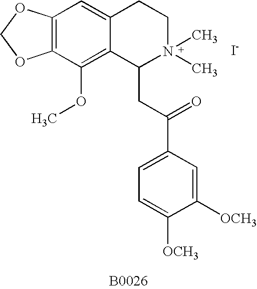

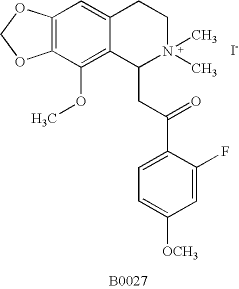

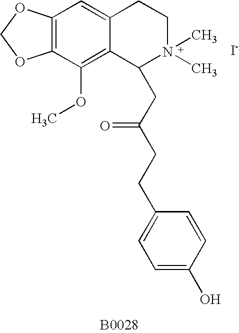

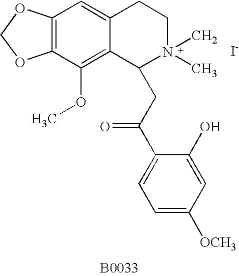

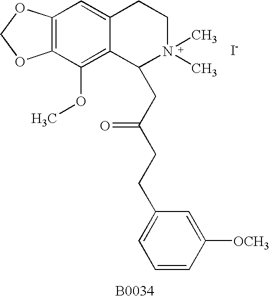

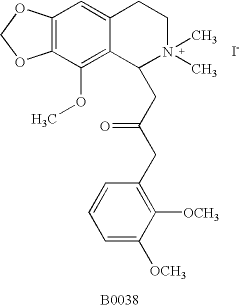

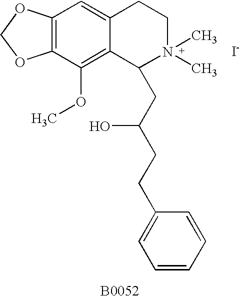

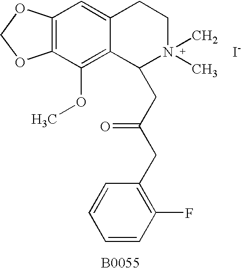

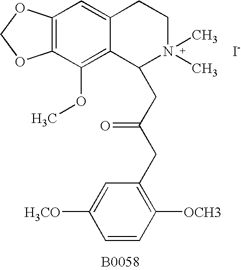

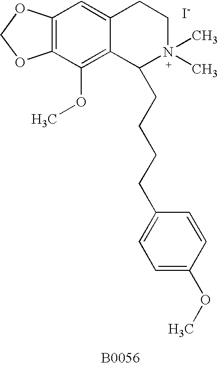

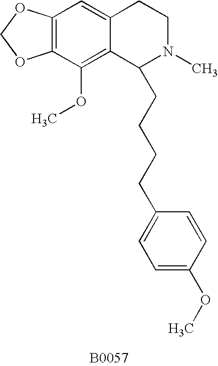

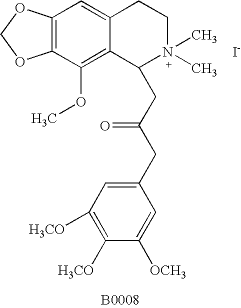

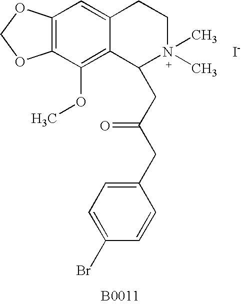

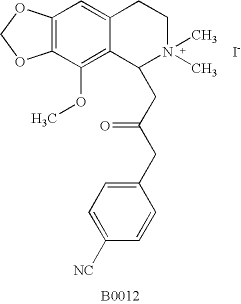

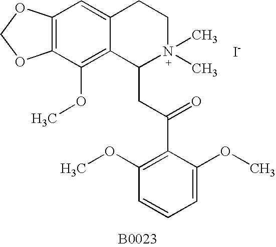

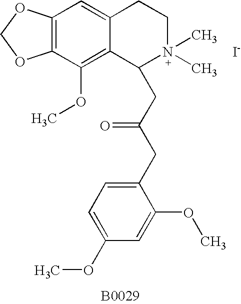

### Example 1

### MOR Agonist Activity

Using GTPγS Binding Assay

To assess the mu opiate receptor (MOR) agonist activity of positive compounds from the FLNA screening, compounds were tested in a [³⁵S]GTPγS binding assay using striatal membranes. Our previous study has shown that in striatal membranes, activation of MOR leads to an increase in [³⁵S]GTPγS binding to Gαo (Wang et al., 2005 Neuroscience 135:247-261).

Striatal tissue was homogenized in 10 volumes of ice cold 25 mM HEPES buffer, pH 7.4, which contained 1 mM EGTA, 100 mM sucrose, 50 μg/ml leupeptin, 0.04 mM PMSF, 2 μg/ml soybean trypsin inhibitor and 0.2% 2-mercaptoethanol. The homogenates were centrifuged at 800×g for 5 minutes and the supernatants were centrifuged at 49,000×g for 20 minutes. The resulting pellets were suspended in 10 volume of reaction buffer, which contained 25 mM HEPES, pH 7.5, 100 mM NaCl, 50 μg/ml leupeptin, 2 μg/ml soybean trypsin inhibitor, 0.04 mM PMSF and 0.02% 2-mercaptomethanol.

The resultant striatal membrane preparation (200 μg) was admixed and maintained (incubated) at 30° C. for 5 minutes in reaction buffer as above that additionally contained 1 mM MgCl₂ and 0.5 nM [³⁵S]GTPγS (0.1 μCi/assay, PerkinElmer Life and Analytical Sciences) in a total volume of 250 μl and continued for 5 minutes in the absence or presence of 0.1-10 μM of an assayed compound of interest. The reaction was terminated by dilution with 750 μl of ice-cold reaction buffer that contained 20 mM MgCl₂ and 1 mM EGTA and immediate centrifugation at 16,000×g for 5 minutes.

The resulting pellet was solubilized by sonicating for 10 seconds in 0.5 ml of immunoprecipitation buffer containing 0.5% digitonin, 0.2% sodium cholate and 0.5% NP-40. Normal rabbit serum (1 μl) was added to 1 ml of lysate and incubated at 25° C. for 30 minutes. Nonspecific immune complexes were removed by incubation with 25 μl of protein A/G-conjugated agarose beads at 25° C. for 30 minutes followed by centrifugation at 5,000×g at 4° C. for 5 minutes. The supernatant was divided and separately incubated at 25° C. for 30 minutes with antibodies raised against Gαo proteins (1:1,000 dilutions).

The immunocomplexes so formed were collected by incubation at 25° C. for 30 minutes with 40 μl of agarose-conjugated protein A/G beads and centrifugation at 5,000×g at 4° C. for 5 minutes. The pellet was washed and suspended in buffer containing 50 mM Tris-HCl, pH 8.0, and 1% NP-40. The radioactivity in the suspension was determined by liquid scintillation spectrometry. The specificity of MOR activation of [³⁵S]GTPγS binding to Gαo induced by a selective compound was defined by inclusion of 1 μM β-funaltrexamine (β-FNA; an alkylating derivative of naltrexone that is a selective MOR antagonist). DAMGO (H-Tyr-D-Ala-Gly-N-MePhe-Gly-OH; 1 or 10 μM) was used as a positive control.

The results of this study are shown in the Table below.

FLNA-Binding Compound MOR Agonist Activity

| FLNA- | Concentration of FLNA-Binding Compound as Agonist | | | | | |
|---|---|---|---|---|---|---|
| Binding | | | 1 μM + | % DAMGO | % DAMGO | % DAMGO + |
| Compound | 0.1 μM | 1 μM | BFNA | (0.1 μM) | (1 μM) | BFNA |
| 5009 | 128.5% | 270.4% | 87.5% | 66.9% | 83.2% | 181.5% |
| B0001 | 128.2% | 202.3% | 28.0% | 77.4% | 74.9% | 43.1% |
| B0002 | 165.7% | 219.0% | 101.4% | 100.0% | 81.1% | 156.0% |
| B0003 | 103.0% | 131.1% | 18.6% | 59.9% | 47.4% | 29.0% |
| B0004 | 170.3% | 231.7% | 72.0% | 102.8% | 85.8% | 110.0% |
| B0005 | 89.2% | 110.4% | 45.1% | 50.5% | 42.6% | 68.6% |
| B0006 | 77.0% | 131.3% | 18.6% | 44.8% | 47.5% | 29.0% |
| B0007 | 168.3% | 223.3% | 64.5% | 95.3% | 86.1% | 98.2% |
| B0008 | 148.3% | 264.1% | 46.0% | 84.0% | 101.9% | 70.0% |
| B0009 | 144.4% | 219.9% | 119.4% | 81.8% | 84.8% | 181.7% |
| B0010 | 132.9% | 184.4% | 152.0% | 75.3% | 71.1% | 231.4% |
| B0011 | 158.6% | 212.6% | 78.0% | 95.7% | 78.7% | 120.0% |
| B0012 | 167.4% | 212.0% | 145.1% | 97.8% | 79.4% | 278.5% |
| B0013 | 51.4% | 154.1% | 34.4% | 29.1% | 59.4% | 52.4% |
| B0014 | 166.6% | 250.5% | 44.3% | 98.5% | 93.7% | 67.1% |
| B0016 | 167.7% | 213.6% | 72.2% | 99.2% | 79.9% | 109.4% |
| B0017 | 99.6% | 122.0% | 49.6% | 58.9% | 45.6% | 75.2% |
| B0018 | 118.8% | 143.0% | 45.6% | 70.3% | 53.5% | 69.1% |
| B0019 | 101.0% | 256.5% | 81.4% | 59.7% | 96.0% | 123.3% |
| B0020 | 51.6% | 181.6% | 24.9% | 30.1% | 68.0% | 47.8% |
| B0021 | 126.9% | 256.4% | 42.9% | 75.9% | 104.7% | 123.6% |
| B0022 | 131.9% | 182.7% | 45.8% | 78.9% | 74.6% | 132.0% |
| B0023 | 166.1% | 245.3% | 28.4% | 99.4% | 100.1% | 81.8% |
| B0024 | 155.8% | 285.9% | 20.2% | 93.2% | 116.7% | 58.2% |
| B0025 | 159.6% | 234.6% | 137.7% | 96.3% | 86.8% | 211.8% |
| B0026 | 152.0% | 233.3% | 28.8% | 88.8% | 87.4% | 55.3% |
| B0027 | 140.9% | 266.9% | 21.6% | 82.3% | 100.0% | 41.5% |
| B0028 | 199.1% | 357.7% | 55.0% | 103.5% | 131.0% | 125.3% |
| B0029 | 171.9% | 210.3% | 17.6% | 89.4% | 77.0% | 40.1% |
| B0030 | 107.2% | 276.1% | 90.1% | 62.6% | 103.4% | 172.9% |
| B0031 | 210.8% | 272.0% | 28.8% | 109.6% | 99.6% | 65.6% |
| B0032 | 221.1% | 297.7% | 15.6% | 115.0% | 109.0% | 35.5% |
| B0033 | 149.3% | 188.9% | 41.9% | 77.6% | 69.2% | 95.4% |
| B0034 | 122.5% | 235.2% | 41.8% | 71.6% | 88.1% | 80.2% |
| B0035 | 188.0% | 248.7% | 74.2% | 109.8% | 93.2% | 142.4% |
| B0036 | 61.4% | 120.6% | 65.1% | 39.2% | 52.1% | 199.7% |
| B0037 | 119.8% | 186.0% | 106.2% | 76.5% | 80.4% | 325.8% |
| B0038 | 147.5% | 205.3% | 117.1% | 94.2% | 88.7% | 359.2% |
| B0039 | 171.8% | 290.5% | 78.3% | 100.4% | 108.8% | 150.3% |
| B0040 | 146.0% | 243.3% | 55.3% | 93.2% | 105.1% | 169.6% |
| B0041 | 61.6% | 109.3% | 41.9% | 39.3% | 47.2% | 128.5% |
| B0042 | 69.9% | 107.5% | 43.1% | 39.6% | 42.3% | 163.9% |
| B0043 | 74.8% | 248.1% | 166.4% | 42.4% | 97.6% | 632.7% |
| B0044 | 87.3% | 170.0% | 134.6% | 49.4% | 66.9% | 511.8% |
| B0045 | 129.3% | 193.1% | 83.8% | 82.6% | 83.4% | 257.1% |
| B0046 | 99.9% | 141.9% | 90.5% | 63.8% | 61.3% | 277.6% |
| B0047 | 187.8% | 235.6% | 68.4% | 106.3% | 92.6% | 260.1% |
| B0048 | 185.1% | 223.4% | 78.5% | 104.8% | 87.8% | 298.5% |
| B0049 | 181.6% | 364.0% | 133.2% | 102.8% | 143.1% | 506.5% |
| B0050 | 98.2% | 211.0% | 48.8% | 58.1% | 96.4% | 294.0% |
| B0051 | 115.6% | 161.9% | 43.8% | 68.4% | 76.7% | 263.9% |
| B0052 | 98.2% | 151.7% | 40.9% | 58.1% | 69.3% | 246.4% |
| B0053 | 160.2% | 299.8% | 134.3% | 94.8% | 137.0% | 809.0% |
| B0054 | 157.8% | 186.7% | 111.0% | 93.4% | 85.3% | 668.7% |
| B0055 | 162.1% | 338.5% | 117.5% | 91.8% | 133.1% | 446.8% |
| B0056 | 174.7% | 288.8% | 41.8% | 98.9% | 113.6% | 158.9% |

### Example 2

### FITC-NLX-Based FLNA Screening Assay

A. Streptavidin-Coated 96-Well Plates

Streptavidin-coated 96-well plates (Reacti-Bind™ NeutrAvidin™ High binding capacity coated 96-well plate, Pierce-ENDOGEN) are washed three times with 200 μl of 50 mM Tris HCl, pH 7.4 according to the manufacturer's recommendation.

B. N-biotinylated VAKGL Pentapeptide VAKGL) (SEQ ID NO:1)

Bn-VAKGL peptide (0.5 mg/plate) is dissolved in 50 μl DMSO and then added to 4450 μl of 50 mM Tris HCl, pH 7.4, containing 100 mM NaCl and protease inhibitors (binding medium) as well as 500 μl superblock in PBS (Pierce-ENDOGEN) [final concentration for DMSO: 1%].

C. Coupling of Bn-VAKGL Peptides to Streptavidin-Coated Plate

The washed streptavidin-coated plates are contacted with 5 μg/well of Bn-VAKGL (100 μl) for 1 hour (incubated) with constant shaking at 25° C. [50 μl of Bn-VAKGL peptide solution from B+50 μl binding medium, final concentration for DMSO: 0.5%]. At the end of the incubation, the plate is washed three times with 200 pa of ice-cold 50 mM Tris HCl, pH 7.4.

D. Binding of FITC-Tagged

naloxone [FITC-NLX] to VAKGL

Bn-VAKGL coated streptavidin plates are incubated with 10 nM fluorescein isothiocyanate-labeled naloxone (FITC-NLX; Invitrogen) in binding medium (50 mM Tris HCl, pH 7.4 containing 100 mM NaCl and protease inhibitors) for 30 minutes at 30° C. with constant shaking. The final assay volume is 100 μl. At the end of incubation, the plate is washed twice with 100 μl of ice-cold 50 mM Tris, pH 7.4. The signal, bound-FITC-NLX is detected using a DTX-880 multi-mode plate reader (Beckman).

E. Screening of Medicinal Chemistry Analogs

The compounds are first individually dissolved in 25% DMSO containing 50 mM Tris HCl, pH 7.4, to a final concentration of 1 mM (assisted by sonication when necessary) and then plated into 96-well compound plates. To screen the medicinal chemistry analogs (new compounds), each compound solution (1 μl) is added to the Bn-VAKGL coated streptavidin plate with 50 μl/well of binding medium followed immediately with addition of 50 μl of FITC-NLX (total assay volume/well is 100 μl). The final screening concentration for each compound is 10 μM.

Each screening plate includes vehicle control (total binding) as well as naloxone (NLX) and/or naltrexone (NTX) as positive controls. Compounds are tested in triplicate or quadruplicate. Percent inhibition of FITC-NLX binding for each compound is calculated [(Total FITC-NLX bound in vehicle—FITC-NLX bound in compound)/Total FITC-NLX bound in vehicle]×100%]. To assess the efficacies and potencies of the selected compounds, compounds that achieve approximately 60-70% inhibition at 10 μM are screened further at 1 and 0.1 μM concentrations.

The results of this screening assay are shown in the table below.

FLNA Peptide Binding Assay

| | |
|---|---|
| FLNA-binding | Concentration of FLNA-binding Compound |

| Compound | 0.01 μM | 0.1 μM | 1 μM |
|---|---|---|---|
| 5009 | 42.5% | 47.3% | 54.3% |
| B0001 | 37.1% | 48.8% | 54.3% |
| B0002 | 40.2% | 46.4% | 55.0% |
| B0003 | 45.4% | 52.9% | 63.5% |
| B0004 | 38.9% | 50.0% | 54.8% |
| B0005 | 31.8% | 34.8% | 41.7% |
| B0006 | 45.1% | 53.5% | 61.3% |
| B0007 | 43.6% | 53.1% | 57.3% |
| B0008 | 35.5% | 40.3% | 52.8% |
| B0009 | 39.6% | 47.6% | 53.6% |
| B0010 | 39.4% | 43.4% | 50.3% |
| B0011 | 40.9% | 50.3% | 55.8% |
| B0012 | 39.4% | 46.9% | 51.7% |
| B0013 | 25.2% | 35.1% | 43.4% |
| B0014 | 25.7% | 30.9% | 37.8% |
| B0015 | 30.4% | 35.3% | 42.3% |
| B0016 | 27.1% | 33.7% | 41.9% |
| B0017 | 28.3% | 36.6% | 44.6% |
| B0018 | 37.2% | 43.7% | 47.6% |
| B0019 | 34.3% | 41.0% | 49.0% |
| B0020 | 38.1% | 45.5% | 50.6% |
| B0021 | 32.5% | 43.1% | 47.6% |
| B0022 | 34.3% | 40.4% | 45.6% |
| B0023 | 28.5% | 37.8% | 46.4% |
| B0024 | 34.8% | 43.4% | 47.7% |
| B0025 | 41.7% | 49.4% | 56.6% |
| B0026 | 41.1% | 43.3% | 48.2% |
| B0027 | 40.2% | 46.7% | 49.8% |
| B0028 | 38.2% | 42.8% | 49.1% |
| B0029 | 33.4% | 42.9% | 50.2% |
| B0030 | 47.0% | 50.5% | 57.6% |
| B0031 | 36.2% | 44.2% | 50.5% |
| B0032 | 45.1% | 51.3% | 48.9% |
| B0033 | 42.1% | 46.8% | 49.4% |
| B0034 | 49.1% | 54.2% | 59.1% |
| B0035 | 45.4% | 44.7% | 51.0% |
| B0036 | 46.6% | 52.8% | 62.1% |
| B0037 | 47.4% | 53.0% | 52.4% |
| B0038 | 41.2% | 50.1% | 57.0% |
| B0039 | 43.3% | 45.7% | 50.9% |
| B0040 | 40.0% | 53.1% | 57.1% |
| B0041 | 44.0% | 46.8% | 52.8% |
| B0042 | 40.8% | 46.4% | 51.6% |
| B0043 | 30.8% | 39.2% | 46.8% |
| B0044 | 35.2% | 39.5% | 44.4% |
| B0045 | 63.2% | 68.2% | 73.9% |
| B0046 | 42.2% | 50.2% | 55.4% |
| B0047 | 30.7% | 37.6% | 47.1% |
| B0048 | 34.7% | 41.9% | 43.9% |
| B0049 | 32.2% | 40.1% | 47.1% |
| B0050 | 29.2% | 34.5% | 39.8% |
| B0051 | 29.9% | 35.7% | 43.7% |
| B0052 | 30.2% | 39.1% | 44.3% |
| B0053 | 33.1% | 37.3% | 47.6% |
| B0054 | 25.6% | 32.6% | 43.3% |

### Example 3

### Tail-Flick Test

The mouse “tail flick” test was used to assay the relative antinociceptive activity of compositions containing a compound to be assayed.

This assay was substantially that disclosed by Xie et al., 2005 J. Neurosci 25:409-416.

The mouse hot-water tail-flick test was performed by placing the distal third of the tail in a water bath maintained at 52° C. The latency until tail withdrawal from the bath was determined and compared among the treatments. A 10 second cutoff was used to avoid tissue damage. Data are converted to percentage of antinociception by the following formula: (response latency−baseline latency)/(cutoff−baseline latency)×100 to generate dose-response curves. Linear regression analysis of the log dose-response curves was used to calculate the A₅₀ (dose that resulted in a 50% antinociceptive effect) doses and the 95% confidence intervals (CIs). Relative potency was determined as a ratio of the A₅₀ values. The significance of the relative potency and the confidence intervals are determined by applying the t test at p<0.05.

To assess tolerance to the antinociceptive effect, the compound was administered twice daily for 7 days at an A₉₀ dose (dose that results in a 90% antinociceptive effect in the 52° C. warm-water tail-flick test), and the tail-flick test was performed daily after the a.m. dose. A significant reduction in tail-flick latency on subsequent days compared to the Day 1 administration of the A₉₀ dose indicates antinociceptive tolerance.

Orally administered morphine exhibited an A₅₀ value of 61.8 (52.4-72.9) mg/kg, and a mean maximum antinoniception amount of about 43% at 56 mg/kg at about 20 minutes. Orally administered compound 5009 exhibited a mean maximum antinoniception amount of about 30% at 32 mg/kg at about 20 minutes, whereas compound 30040 exhibited a mean maximum antinoniception amount of about 25% at 32 mg/kg at about 20-30 minutes, and compound 30036 exhibited a mean maximum antinoniception amount of about 20% at 32 mg/kg at about 30 minutes.

### Example 4

### Dependence Test

On day 8, 16-20 hours after the last administration of an assay composition, animals were given naloxone to precipitate withdrawal (10 mg/kg, s.c.) before being placed in an observation chamber for 1 hour. A scale adapted from MacRae et al., 1997 Psychobiology 25:77-82 was used to quantify four categories of withdrawal behaviors: “wet dog” shakes, paw tremors, mouth movements, and ear wipes. Scores are summed to yield a total withdrawal score across the 1-hour test.

### Example 5

### Relative Gs/Go Switching

In this set of studies, the rat brain slice organotypic culture methods were modified from those published previously (Adamchik et al., 2000 Brain Res Protoc 5:153-158; Stoppini et al., 1991 J Neurosci Methods 37:173-182). Striatal slices (200 μM thickness) were prepared using a Mcllwain tissue chopper (Mickle Laboratory Engineering Co., Surrey, UK). Slices were carefully transferred to sterile, porous culture inserts (0.4 μm, Millicell-CM) using the rear end of a glass Pasteur pipette. Each culture insert unit contained 2 slices and was placed into one well of the 12-well culture tray. Each well contain 1.5 ml of culture medium composed of 50% MEM with Earl's salts, 2 mM L-glutamine, 25% Earl's balanced salt solution, 6.5 g/l D-glucose, 20% fetal bovine serum, 5% horse serum, 25 mM HEPES buffer, 50 mg/ml streptomycin and 50 mg/ml penicillin. The pH value was adjusted to 7.2 with HEPES buffer.

Cultures were first incubated for 2 days to minimize the impact of injury from slice preparation. Incubator settings throughout the experiment were 36° C. with 5% CO₂. To induce tolerance, culture medium was removed and the culture insert containing the slices was gently rinsed twice with warm (37° C.) phosphate-buffered saline (pH 7.2) before incubation in 0.1% fetal bovine serum-containing culture medium with 100 μM morphine for 1 hour twice daily (at 9-10 AM and 3-4 PM) for 7 days.

Slices were returned to culture medium with normal serum after each drug exposure. Tissues were harvested 16 hours after the last drug exposure by centrifugation.

For determination of MOR-G protein coupling, slices were homogenized to generate synaptic membranes. Synaptic membranes (400 μg) were incubated with either 10 μM oxycodone or Kreb's-Ringer solution for 10 minutes before solubilization in 250 μl of immunoprecipitation buffer (25 mM HEPES, pH 7.5; 200 mM NaCl, 1 mM EDTA, 50 μg/ml leupeptin, 10 μg/ml aprotinin, 2 μg/ml soybean trypsin inhibitor, 0.04 mM PMSF and mixture of protein phosphatase inhibitors). Following centrifugation, striatal membrane lysates were immunoprecipitated with immobilized anti-Gαs/olf or -Gαo conjugated with immobilized protein G-agarose beads. The level of MOR in anti-Gαs/olf or -Gαo immunoprecipitates was determined by Western blotting using specific anti-MOR antibodies.

To measure the magnitude of MOR-mediated inhibition of cAMP production, brain slices were incubated with Kreb's-Ringer (basal), 1 μM DAMGO, 1 μM forskolin or 1 μM DAMGO+1 μM forskolin for 10 minutes at 37° C. in the presence of 100 μM of the phosphodiesterase inhibitor IBMX. Tissues were homogenized by sonication and protein precipitated with 1M TCA. The supernatant obtained after centrifugation was neutralized using 50 mM Tris, pH 9.0. The level of cAMP in the brain lysate was measured by a cAMP assay kit (PerkinElmer Life Science, Boston) according to manufacturer's instructions.

| | | | Gs/Go-Coupled |
|---|---|---|---|
| Condition | Gs/olf | Go | Ratio |
| Vehicle | | | |
| Average | 330.7 | 1996.4 | 0.173 |
| SEM | 34.6 | 192.0 | 0.34 |
| Oxycodone, | | | |
| 10 μM | | | |
| Average | 1425.2 | 900.4 | 1.588 |
| SEM | 77.8 | 26.2 | 0.103 |
| B0040, | | | |
| 10 μM | | | |
| Average | 839.0 | 1419.8 | 0.598 |
| SEM | 31.2 | 93.2 | 0.053 |
| B0040, | | | |
| 100 μM | | | |
| Average | 867.2 | 1472.6 | 0.332 |
| SEM | 85.7 | 86.5 | 0.023 |

### Compound Synthesis

A contemplated compound can be readily synthesized. An illustrative synthetic scheme is shown below for the preparation of a first portion of a contemplated compound, with the second portion being added by a reaction of an appropriately substituted methylketone compound. More detailed syntheses are set out hereinafter.

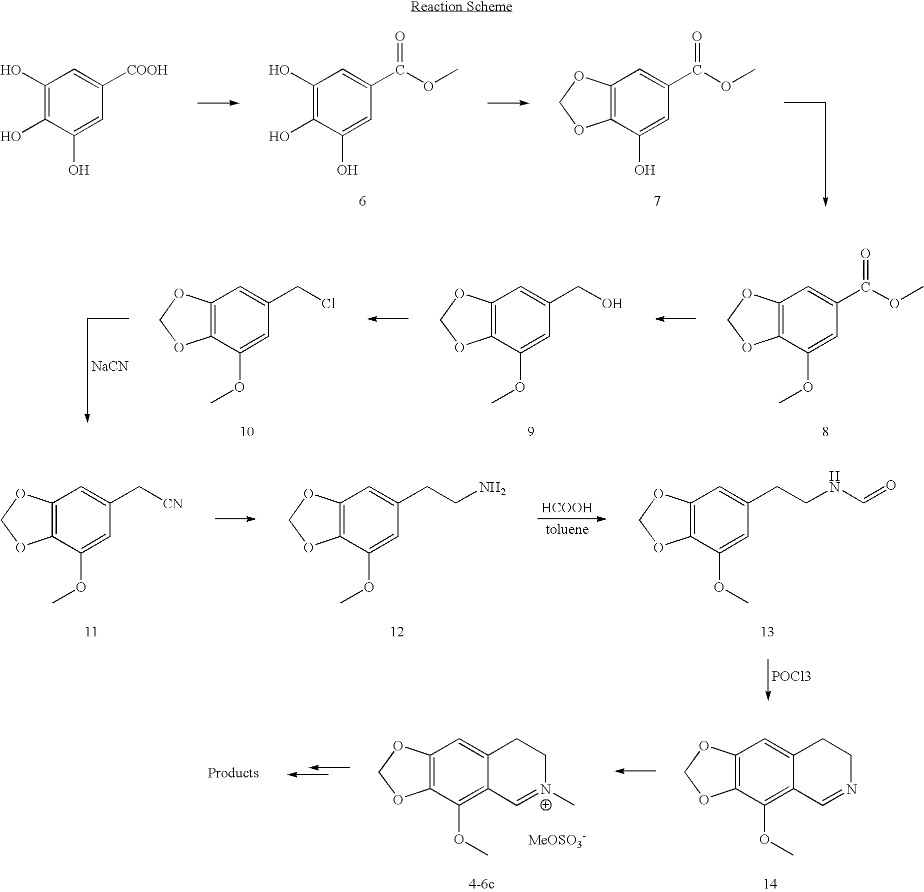

### 1) Synthesis of Compound 4-6c

Synthesis of Compound 6

To a solution of 3,4,5-trihydroxybenzoic acid (3 g, 17.6 mmol) in methanol (30 ml) was added concentrated sulfuric acid (0.9 ml) and the mixture was stirred under reflux for 1.5 hours. The reaction vessel was cooled to room temperature and the reaction mixture was neutralized with saturated sodium bicarbonate solution at 0° C. The organic solvent was removed under reduced pressure. The concentrated residue was dissolved in ethyl acetate, washed with saturated sodium bicarbonate solution and brine, dried over anhydrous Na₂SO₄, concentrated and dried under vacuum to give compound 6 (2.424 g, yield: 74.8%) as a white solid.

Synthesis of Compound 7

To a solution of compound 6 (1 g, 5.43 mmole) in DMSO (25 ml) was added KHCO₃ (0.54 g, 5.43 mmole) followed by dibromomethane (0.4 ml) and the mixture was heated at 60° C. for 1.5 hours under nitrogen. The reaction was cooled and poured into water (50 ml). The mixture was extracted with ether. The organic layers were combined, dried over anhydrous Na₂SO₄ and concentrated to yield a crude oil which was further purified by column chromatography on silica gel (petroleum ether:ethyl acetate=5:1) to yield compound 7 (580 mg, yield: 55%, NMR confirmed) as a white solid.

Synthesis of Compound 8

To a suspension of K₂CO₃ (211 mg, 1.53 mmol) in DMSO (5 mL) was added a solution of compound 7 (200 mg, 1.02 mmol) in DMSO (5 mL) and the mixture was stirred at room temperature for 30 minutes. CH₃I (217 mg, 1.53 mmol) was added, and the reaction mixture was stirred for another 4 hours. DMSO was removed under reduced pressure and the residue obtained was extracted with ethyl acetate. The organic layer was dried over anhydrous Na₂SO₄ and concentrated under reduced pressure to yield compound 8 (180 mg, yield: 84%, NMR confirmed) as a yellow solid.

Synthesis of Compound 9

Compound 8 (56 mg, 0.27 mmol) was added dropwise to a suspension of LiAlH₄ (40 mg, 1.08 mmol) in THF (5 mL) at 0° C. The reaction mixture was stirred at 0° C. for 30 minutes, followed by stirring at room temperature for 1 hour. The reaction was quenched with cold water (10 ml) and extracted with ethyl acetate. The organic layer was dried over anhydrous Na₂SO₄ and concentrated under reduced pressure to yield compound 9 (48 mg, yield: 97.7%, NMR confirmed) as yellow oil.

Synthesis of Compound 10

To a mixture of Compound 9 (1.15 g, 5.5 mmol) in THF (20 ml) was added SOCl₂ (0.8 ml) and the reaction mixture was stirred at room temperature for 3.5 hours. The reaction was quenched with water and extracted with ethyl acetate. The organic layer was dried over Na₂SO₄ and concentrated under reduced pressure to give compound 10 (1.056 g, yield: 95.5%, NMR confirmed) as a yellow solid.

Synthesis of Compound 11

A solution of compound 10 (1.056 g, 5.25 mmol) and NaCN (0.52 g, 10.5 mmol) in DMF (25 mL) was stirred at 100° C. for 4 hours whereupon the color of the reaction mixture changed from yellow to black. The reaction was diluted with water and extracted with ethyl acetate three times. The combined layers of ethyl acetate were washed with water, dried over anhydrous Na₂SO₄ and concentrated under reduced pressure to give compound II (630 mg, yield: 62.8%, NMR confirmed) as a brown solid.

Synthesis of Compound 12

To a mixture of EtOH (20 mL), water (8 mL) and 1N HCl (2 mL) was added PtO₂ (200 mg) and compound II (630 mg, 3.56 mmol) and the reaction mixture was hydrogenated overnight (about 18 hours) under 40 psi of H₂ at room temperature. TLC showed the reaction was complete. The solution was concentrated under reduced pressure. To the residue was added water and 1M NaOH till the solution reached pH 13˜14. The resulting mixture was extracted with CH₂Cl₂. The organic layer was dried over Na₂SO₄ and concentrated to give compound 12 (670 mg, yield: 96.5%, NMR confirmed) as brown oil.

Synthesis of Compound 13

A mixture of compound 12 (670 mg, 3.44 mmol) and formic acid (0.62 mL) in toluene (20 mL) was refluxed for 4 hours following which the reaction was partitioned between water and toluene and the aqueous layer was extracted with toluene three times. The combined organic layers were washed with water and brine and concentrated to obtain compound 13 (539 mg, yield: 70.3%, NMR confirmed) as a brown solid.

Synthesis of Compound 14

To a solution of compound 13 (500 mg, 2.24 mmol) in CH₂Cl₂ (10 ml) was added POCl₃ (0.5 ml) and the reaction mixture was refluxed at 75° C. for 3 hours. The reaction mixture was concentrated under reduced pressure and to the residue was added water (20 ml), toluene (20 ml) and 20% NaOH (5 ml). The mixture was stirred at 100° C. for 1 h and cooled. The layers were separated and the aqueous layer was extracted with ethyl acetate. The combined organic layers were washed with water and brine and concentrated to obtain the crude product (394 mg) which was further purified by column chromatography over silica gel to obtain compound 14 (320 mg, yield: 69.7%, NMR confirmed) as a white solid.

Synthesis of Compound 4-6c

A mixture of compound 14 (200 mg, 0.98 mmol) and dimethylsulfate (0.1 mL) in toluene (10 mL) was stirred overnight (about 18 hours) at room temperature. The reaction mixture was filtered and the deposited crystals were washed with toluene and dried to afford compound 4-6c (148 mg, yield: 45.6%, NMR and LC-MS confirmed) as a yellow solid.

### Synthesis of Compound 6810

Synthesis of Compound Pre-6810

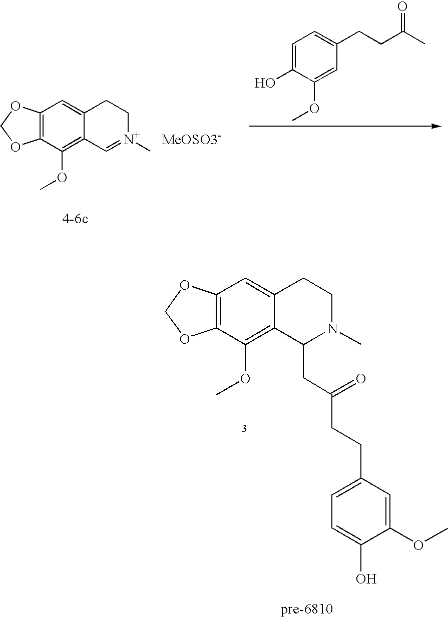

A solution of EtONa was prepared by adding sodium (6.9 mg) to EtOH (2 mL) at 0° C. and after the pieces of sodium had disappeared, this solution was added dropwise to a solution of compound 4-6c (50 mg, 15.1 mmol) in EtOH (5 mL) at 0° C. and the mixture was stirred for 20 min. 4-(4-hydroxy-3-methoxyphenyl)-butan-2-one (44 mg, 22.6 mmol) was added and the reaction mixture was stirred overnight (about 18 hours) at room temperature. 1N HCl (5 mL) was added to the reaction mixture which was concentrated to remove the solvent. The acidic solution was washed with Et₂O and the pH was adjusted to pH 8 with NaHCO₃. The resulting mixture was extracted with CH₂Cl₂. The CH₂Cl₂ layer was dried over anhydrous Na₂SO₄ and concentrated under reduced pressure to obtain compound pre-6810 (31 mg, yield: 49.7%, NMR confirmed).

Synthesis of Compound 6810

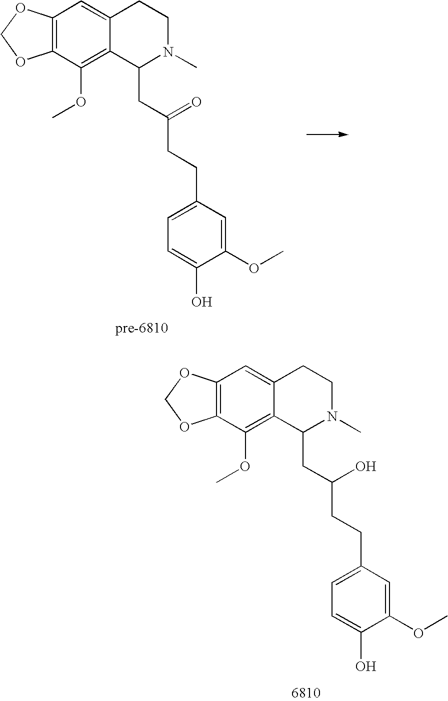

A mixture of compound pre-6810 (74 mg, 0.18 mmol), NaBH₄ (27 mg, 0.72 mmol) and CH₃OH (6 mL) was stirred in an ice bath for 30 minutes and then at room temperature for 1.5 hours. The completion of the reaction was observed by Thin Layer Chromatography (TLC). The reaction mixture was concentrated under reduced pressure, diluted with water and extracted with CH₂Cl₂. The organic layer was dried over anhydrous Na₂SO₄ and concentrated under reduced pressure to give compound 6810 (58 mg, yield: 78%, MS confirmed) as a yellow glue.

### Synthesis of Compound B0001

Synthesis of Compound pre-B0001

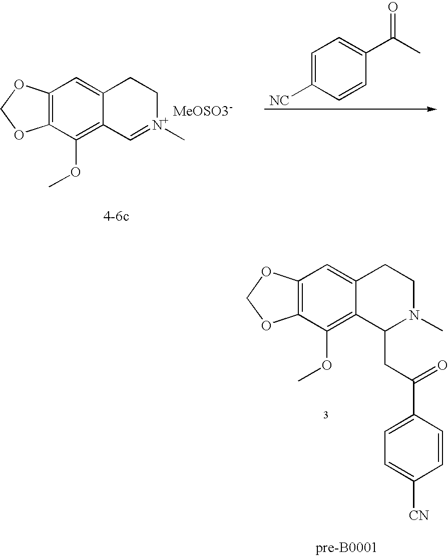

A solution of EtONa was prepared by adding sodium (150 mg) to EtOH (8 mL) at 0° C. and after the pieces of sodium had disappeared, this solution was added dropwise to a solution of compound 4-6c (400 mg, 1.2 mmol) in EtOH (12 mL) at 0° C. and the mixture was stirred for 20 minutes. A solution of 4-acetyl-benzonitrile (263 mg, 1.8 mmol) in EtOH (1 ml) was added and the mixture was stirred overnight (about 18 hours) at room temperature. The precipitate formed was filtered to give compound pre-B0001 (200 mg, yield: 45%, NMR confirmed).

Synthesis of Compound B0001

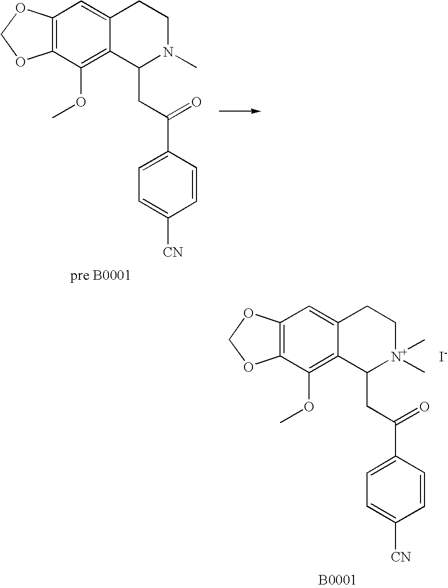

To a solution of pre-MED-B0001 (203 mg, 0.558 mmol) in toluene (12 mL) was added CH₃I (0.4 mL) and the reaction mixture was stirred overnight (about 18 hours) at room temperature. The precipitate formed was collected by filtration and dried to obtain compound B0001 (190 mg, yield: 67.3%) as a yellow solid.

### Synthesis of Compound B0003

Synthesis of Compound pre-B0003

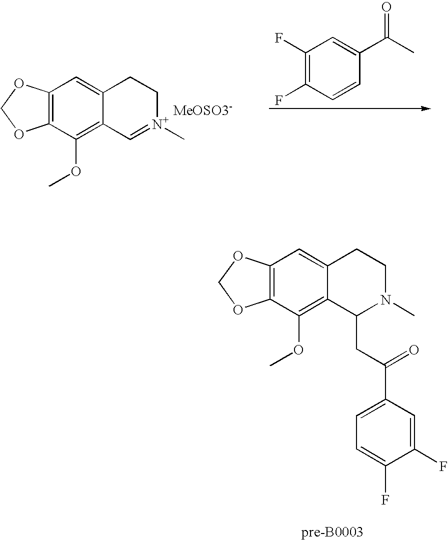

A solution of EtONa was prepared by adding sodium (41.7 mg) to EtOH (10 mL) at 0° C. and after the pieces of sodium had disappeared, this solution was added dropwise to a solution of compound 4-6c (300 mg, 0.906 mmol) in EtOH (8 mL) at 0° C. and the mixture was stirred for 20 minutes. 1-(3,4-difluorophenyl)-ethanone (212 mg, 1.359 mmol) was added and the mixture was stirred at room temperature for 2 days. To the reaction mixture was added 1N HCl (7 mL) and the reaction mixture was concentrated. The acidic solution was washed with Et₂O and the pH was adjusted to pH 13-14 with 1M NaOH. The resulting mixture was extracted with CH₂Cl₂. The CH₂Cl₂ layer was dried over anhydrous Na₂SO₄ and concentrated under reduced pressure to obtain the crude product which was further purified to give compound pre-B0003 (80 mg, yield: 23.5%).

Synthesis of Compound B0003

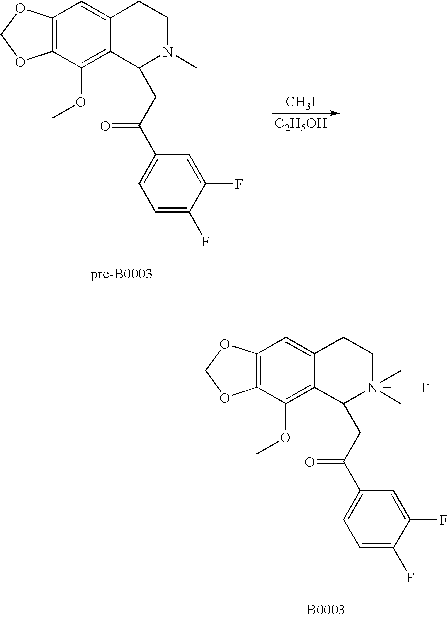

To a solution of compound pre-B0003 (92 mg, 0.25 mmol) in toluene (6 mL) was added CH₃I (0.2 mL) and the reaction mixture was stirred overnight (about 18 hours) at room temperature. The precipitate formed was collected by filtration and dried to obtain compound B0003 (92 mg, yield: 71.2, NMR and MS confirmed) as a yellow solid. The product (60 mg) was recrystallized in ethanol. After the compound had dissolved in hot ethanol, the solution was cooled to room temperature and filtered to give compound B0003 (42 mg, yield: 70%, NMR confirmed) as a white solid.

### Synthesis of Compound B0004

Synthesis of Compound pre-B0004

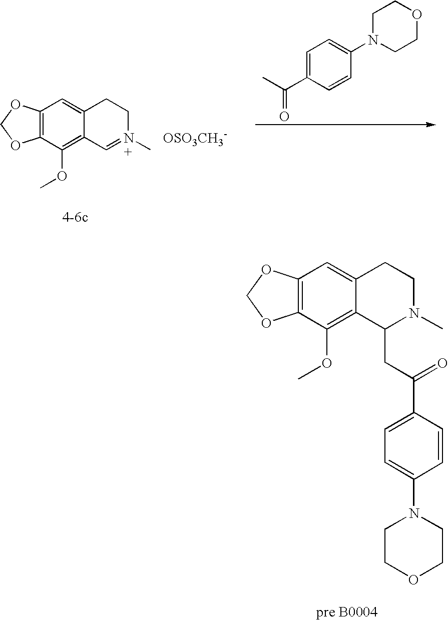

A solution of EtONa was prepared by adding sodium (31 mg) to EtOH (4 mL) at 0° C. and after the pieces of sodium had disappeared, this solution was added dropwise to a solution of compound 4-6c (221 mg, 0.67 mmol) in EtOH (5 mL) at 0° C. and the mixture was stirred for 20 minutes. 1-(4-Morpholinophenyl)-ethanone (205 mg, 1 mmol) in EtOH (1 mL) was added and the reaction mixture was stirred overnight (about 18 hours) at room temperature. To the reaction mixture was added 1N HCl (5 mL) and the reaction mixture was concentrated. The acidic solution was washed with Et₂O and the pH was adjusted to pH 13-14 with 1N NaOH. The resulting mixture was extracted with CH₂Cl₂. The CH₂Cl₂ layer was dried over anhydrous Na₂SO₄ and concentrated under reduced pressure to obtain compound B0004 (228 mg, yield: 80%, NMR confirmed) as a yellow solid.

Synthesis of Compound B0004

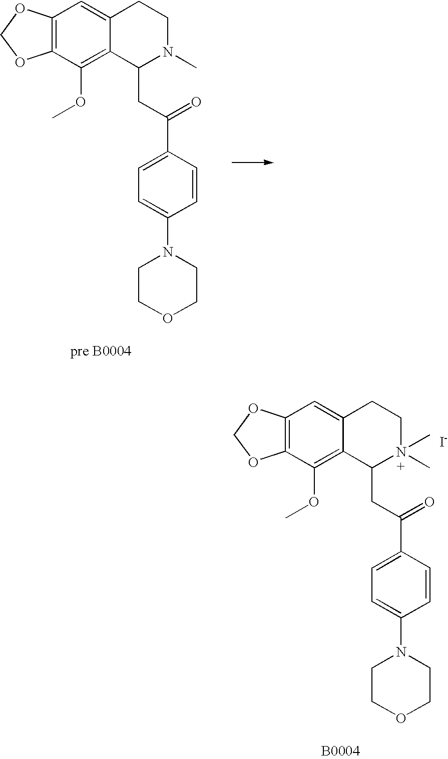

To a solution of pre-B0004 (218 mg, 0.514 mmol) in toluene (10 mL) was added CH₃I (0.25 mL) and the mixture was stirred overnight (about 18 hours) at room temperature. The precipitate formed was collected by filtration and dried to obtain the crude product (234 mg) which was recrystallized in EtOH (3 mL) to obtain compound B0004 (148 mg, yield: 51.7%, NMR and MS confirmed) as a yellow solid.

Synthesis of Compound 0005

Synthesis of Compound pre-B0005

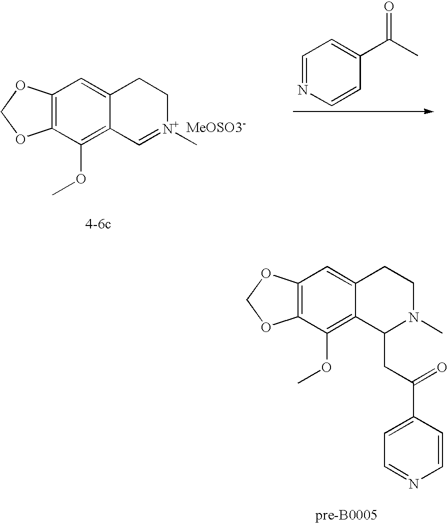

A solution of EtONa was prepared by adding sodium (25 mg) to EtOH (5 mL) at 0° C. and after the pieces of sodium had disappeared, this solution was added dropwise to a solution of compound 4-6c (180 mg, 0.54 mmol) in EtOH (5 mL) at 0° C. and the mixture was stirred for 20 minutes. 1-Pyridin-4-yl-ethanone (98 mg, 0.81 mmol) was added and the reaction mixture was stirred at room temperature for 2 days. To the reaction mixture was added 1N HCl (5 mL) and the reaction mixture was concentrated. The acidic solution was washed with Et₂O and the pH was adjusted to pH 13-14 with 1M NaOH. The resulting mixture was extracted with CH₂Cl₂. The CH₂Cl₂ layer was dried over anhydrous Na₂SO₄ and the solution was concentrated under reduced pressure to obtain the crude product which was passed through an Al₂O₃ column to obtain pre-MED-B0005 (19 mg, yield: 10.3%).

### Synthesis of Compound B0006

Synthesis of Compound pre-B0006

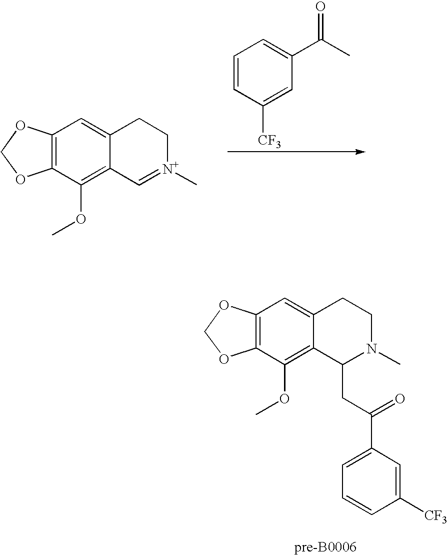

A solution of EtONa was prepared by adding sodium (28 mg) to EtOH (7 mL) at 0° C. and after the pieces of sodium had disappeared, this solution was added dropwise to a solution of compound 4-6c (200 mg, 0.604 mmol) in EtOH (8 mL) at 0° C. and the mixture was stirred for 20 minutes. 4-(4-methoxyphenyl)-butan-2-one (161.2 mg, 0.906 mmol) was added and the reaction mixture was stirred overnight (about 18 hours) at room temperature. To the reaction mixture was added 1N HCl (5 mL) and the reaction was concentrated. The acidic solution was washed with Et₂O and the pH was adjusted to pH 13-14 with 1M NaOH. The resulting mixture was extracted with CH₂Cl₂. The CH₂Cl₂ layer was dried over anhydrous Na₂SO₄ and concentrated under reduced pressure to obtain the crude product which was passed through an Al₂O₃ column to obtain pre-0006 (132 mg, yield: 55%, NMR and MS confirmed).

Synthesis of Compound 30006

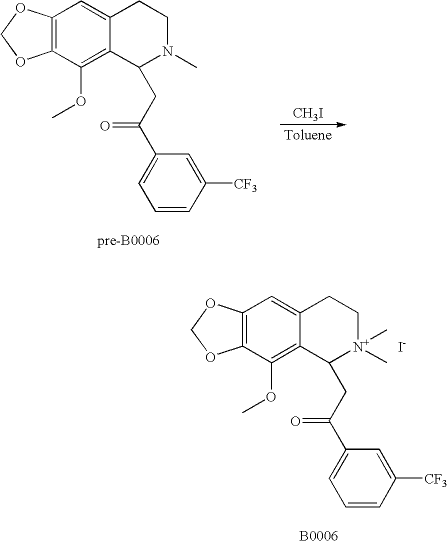

To a solution of pre-B0006 (20 mg, 0.049 mmol) in toluene (2 mL) was added CH₃I (0.1 mL) and the reaction mixture was stirred overnight (about 18 hours) at room temperature. The precipitate formed was collected by filtration and dried to obtain compound B0006 (10 mg, yield: 37.2%) as a yellow solid.

### Synthesis of Compound B0007

Synthesis of Compound pre-B0007

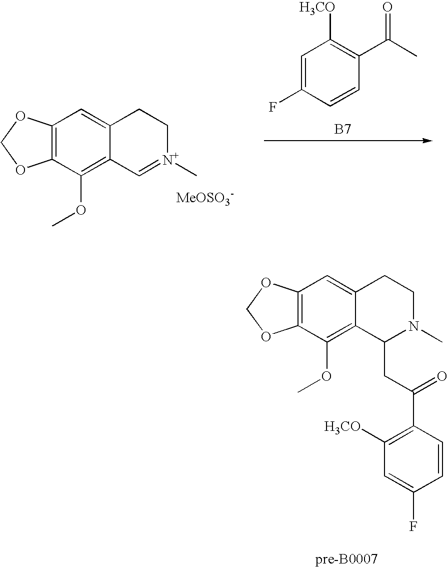

A solution of EtONa was prepared by adding sodium (50 mg) to EtOH (7 mL) at 0° C. and after the pieces of sodium had disappeared, this solution was added dropwise to a solution of compound 4-6c (180 mg, 0.54 mmol) in EtOH (5 mL) at 0° C. and the mixture was stirred for 20 minutes. 1-(4-fluoro-2-methoxyphenyl)—ethanone (137 mg, 0.81 mmol) was added and the reaction mixture was stirred at room temperature for 2 days. To the reaction mixture was added 1N HCl (5 mL) and the reaction was concentrated. The acidic solution was washed with Et₂O and the pH was adjusted to pH 13-14 with 2N NaOH. The resulting mixture was extracted with CH₂Cl₂. The CH₂Cl₂ extract was dried over anhydrous Na₂SO₄ and concentrated under reduced pressure to obtain the crude product which was passed through an Al₂O₃ column to obtain compound pre-B0007 (115 mg, yield: 55%).

Synthesis of Compound B0007

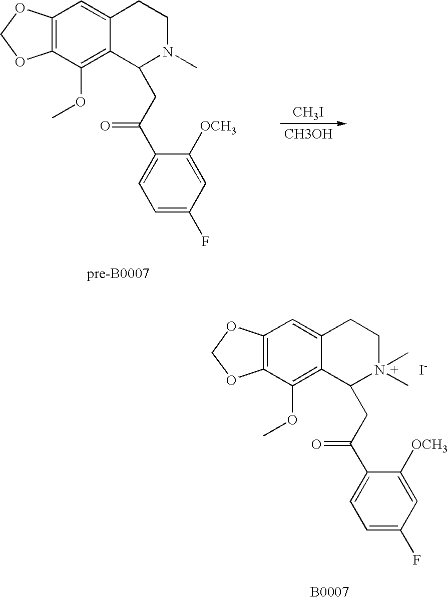

To a solution of pre-MED-B0007 (115 mg, 0.297 mmol) in toluene (8 mL) was added CH₃I (0.3 mL) and the reaction mixture was stirred overnight (about 18 hours) at room temperature. The precipitate formed was collected by filtration and dried to obtain compound B0007 (112 mg, yield: 71.4%, NMR and MS confirmed) as a white solid. The crude product (47 mg) was recrystallized in ethanol by heating. After the compound was dissolved in hot ethanol, the solution was cooled to room temperature and filtered to give compound B0007 (15 mg, yield: 31.9%) as a white solid.

### Synthesis of Compound B0015

Synthesis of Compound pre-B0015

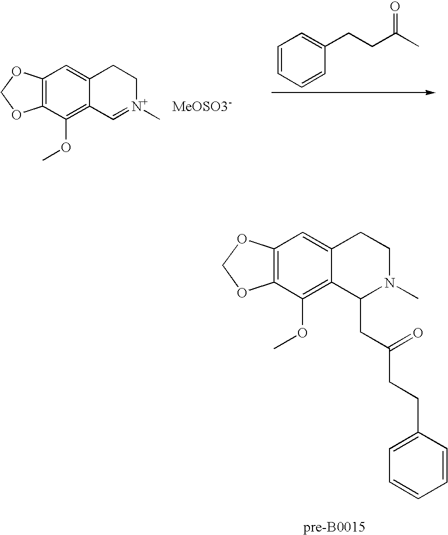

A solution of EtONa was prepared by adding sodium (28 mg) to EtOH (7 mL) at 0° C. and after the pieces of sodium had disappeared, this solution was added dropwise to a solution of compound 4-6c (200 mg, 0.604 mmol) in EtOH (8 mL) at 0° C. and the mixture was stirred for 20 minutes. 4-phenyl-2-butanone (134 mg, 0.906 mmol) was added and the reaction mixture was stirred at room temperature for 2 days. To the reaction mixture was added 1N HCl (5 mL) and the reaction was concentrated. The acidic solution was washed with Et₂O and the pH of the solution was adjusted to pH 13 with 1M NaOH. The resulting mixture was extracted with CH₂Cl₂. The CH₂Cl₂ layer was dried over anhydrous Na₂SO₄ and concentrated under reduced pressure to obtain the crude product which was further purified by column chromatography over Al₂O₃ to give compound pre-B0015 (125 mg, yield: 56.4%, NMR confirmed) as white oil.

Synthesis of Compound B0015

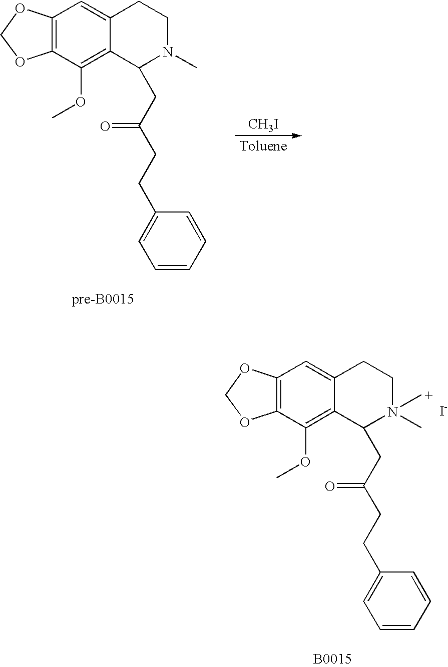

To a solution of pre-B0015 (120 mg, 0.327 mmol) in toluene (10 mL) was added CH₃I (0.3 mL) and the mixture was stirred overnight (about 18 hours) at room temperature. The reaction mixture was filtered and the deposited crystals were washed with toluene and dried to afford compound B0015 (142 mg, yield: 85.3%, NMR confirmed) as a white solid. The product (100 mg) was recrystallized in ethanol. After the compound had dissolved in ethanol, the solution was cooled to room temperature and filtered to give compound B0015 (72 mg, yield: 72%, NMR confirmed) as a white solid.

### Synthesis of Compound B0016

Synthesis of Compound pre-B0016

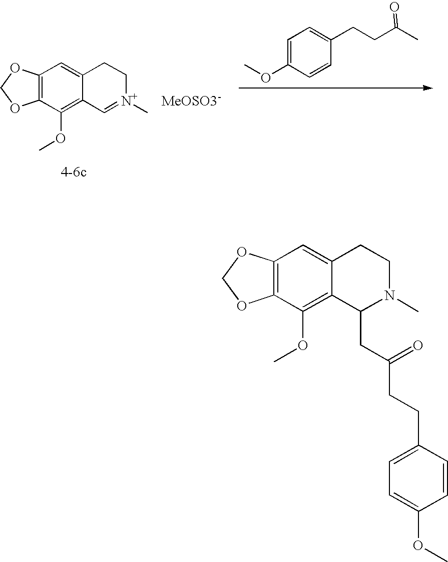

A solution of EtONa was prepared by adding sodium (69.5 mg) to EtOH (9 mL) at 0° C. and after the pieces of sodium had disappeared, this solution was added dropwise to a solution of compound 4-6c (500 mg, 1.51 mmol) in EtOH (11 mL) at 0° C. and the mixture was stirred for 20 minutes. 4-(4-methoxyphenyl)-butan-2-one (403 mg, 2.265 mmol) was added and the reaction mixture was stirred at room temperature for 2 days. To the reaction mixture was added 1N HCl (5 mL) and the reaction mixture was concentrated. The acidic solution was washed with Et₂O and the pH was adjusted to pH 13-14 with 1M NaOH. The resulting mixture was extracted with CH₂Cl₂. The CH₂Cl₂ layer was dried over anhydrous Na₂SO₄ and the solution was concentrated under reduced pressure to obtain the crude product which was passed through Al₂O₃ column to obtain pre-B0016 (363 mg, yield: 60.6, NMR confirmed).

Synthesis of Compound B0016

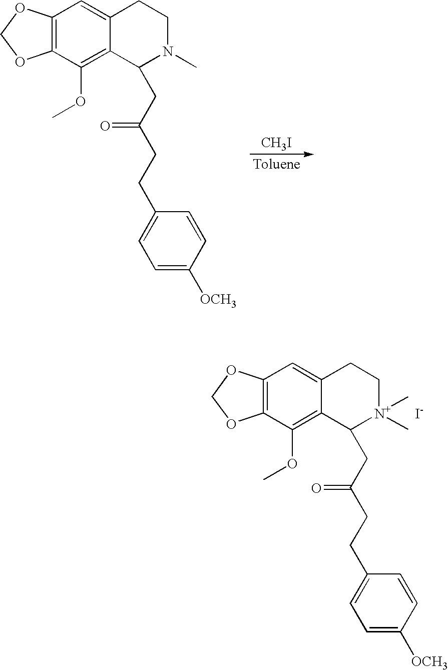

To a solution of pre-MED-B0016 (120 mg, 0.302 mmol) in toluene (10 mL) was added CH₃I (0.2 mL) and the mixture was stirred overnight (about 18 hours) at room temperature. The precipitate formed was collected by filtration and dried to obtain compound 0016 (116 mg, NMR and MS confirmed) as a light yellow solid.

### Synthesis of Compound B0017

Synthesis of Compound pre-B0017

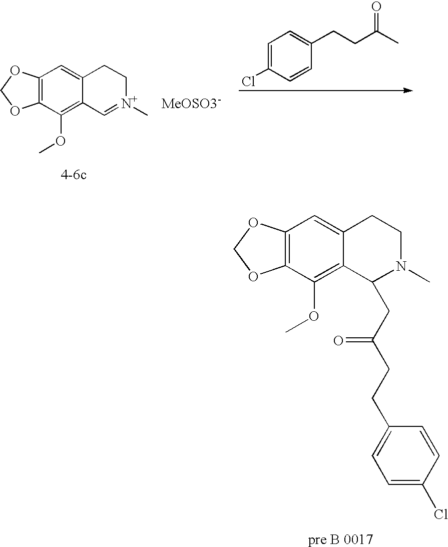

A solution of EtONa was prepared by adding sodium (69 mg) to EtOH (10 mL) at 0° C. and after the pieces of sodium had disappeared, this solution was added dropwise to a solution of compound 4-6c (500 mg, 1.51 mmol) in EtOH (8 mL) at 0° C. and the mixture was stirred for 20 minutes. 4-(4-chlorophenyl)-butan-2-one (412 mg, 2.265 mmol) was added and the reaction mixture was stirred overnight (about 18 hours) at room temperature. To the reaction mixture was added 1N HCl (5 mL) and the reaction mixture was concentrated. The acidic solution was washed with Et₂O and the pH was adjusted to pH 13-14 with 1M NaOH. The resulting mixture was extracted with CH₂Cl₂. The CH₂Cl₂ layer was dried over anhydrous Na₂SO₄ and the solution was concentrated under reduced pressure to obtain the crude product which was passed through Al₂O₃ column to obtain compound pre-B0017 (342 mg, yield: 56.5%, NMR confirmed) as white oil.

### Synthesis of Compound B0036-7

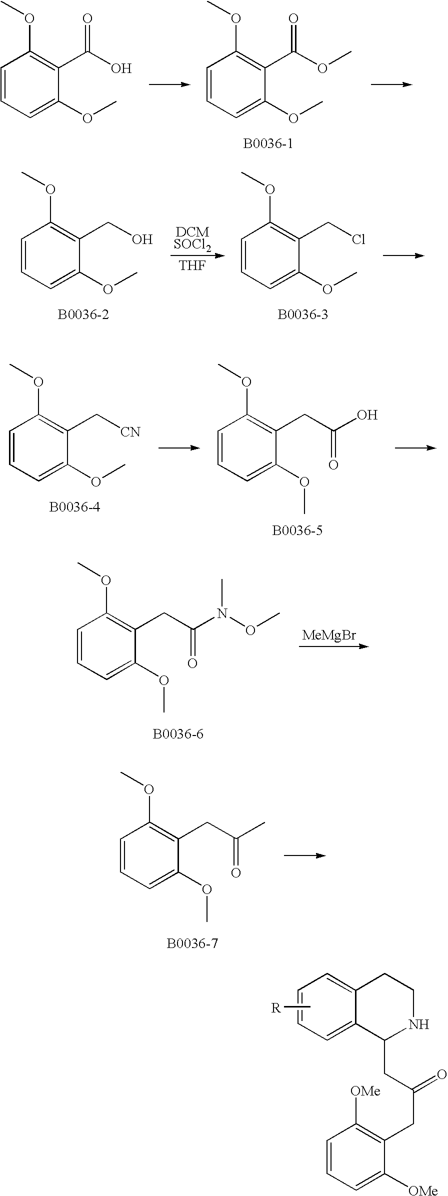

Synthesis of Compound B0036-1

2,6-Dimethoxybenzoic acid (5.0 g, 27.5 mmol) was dissolved in sulfuryl dichloride (20 ml) and DMF (0.05 ml) was added. The mixture was stirred at room temperature for 2 hours following which sulfuryl dichloride was removed under reduced pressure. To the concentrated residue was added methanol (60 ml) and the mixture was stirred at room temperature for 0.5 hours. Methanol was removed under reduced pressure and the residue was purified by column chromatography over silica gel to give compound B0036-1 (5.39 g, Yield: 100%, NMR confirmed) as a white solid.

Synthesis of Compound B0036-2

Compound B0036-1 (5.0 mg, 27.5 mmol) was dissolved in THF (40 ml) and added to LiAlH₄ (2.09 g, 55 mmol) in THF (40 ml) at 0° C. The mixture was stirred at room temperature for 0.5 hours following which ice water was added and the solution was extracted with ethyl acetate. The organic layer was dried over anhydrous Na₂SO₄ and concentrated to obtain compound B0036-2 (4.42 g, Yield: NMR confirmed) as a white solid.

Synthesis of Compound B0036-3

Compound B0036-2 (8.84 g, 52.6 mmol) was dissolved in THF (100 ml) and sulfuryl dichloride (20 ml) was added. The mixture was stirred at room temperature for 2 hours following which ice water was added and the solution was extracted with ethyl acetate. The organic layer was dried over anhydrous Na₂SO₄ and concentrated to obtain the crude compound B0036-3 (8.5 g, Yield: 86%) as a yellow solid.

Synthesis of Compound B0036-4

To a solution of compound B0036-3 (8.5 g, 45.7 mmol) in DMF (50 mL) was added NaCN (3.358 g, 69 mmol) and the reaction mixture was stirred at 100° C. for 3 hours. The reaction mixture was cooled to room temperature, and filtered. The filtrate was evaporated to dryness and the residue was partitioned between ethyl acetate and water. The combined organic layers were washed with water and brine, dried over Na₂SO₄, concentrated and purified by column chromatography over silica gel to obtain compound B0036-4 (6.02 g, Yield: 74%, NMR confirmed) as a white solid.

Synthesis of Compound B0036-5

Compound B0036-4 (2.0 g, 11.3 mmol) was dissolved in 50% sulfuric acid (30 ml) and the mixture was heated at reflux for 2 hours. Upon cooling to room temperature, a brown solid crystallized from the solution. The solid was filtered, dissolved in ether and washed with brine. The ether layer was dried over Na₂SO₄ and concentrated to obtain B0036-5 (1.23 g, Yield: 55%, NMR confirmed) as a white, solid.

Synthesis of Compound B0036-6

To a solution of compound B0036-5 (490 mg, 2.50 mmol) in CH₂Cl₂ (10 ml) at 0° C. was added N-methylmorpholine (630 mg, 6.25 mmol) and isobutyl chloroformate (430 mg, 3.15 mmol) and the resulting solution was stirred at 0° C. for 15 min. This was followed by addition of N,O-dimethylhydroxylamine hydrochloride (300 mg, 3.0 mmol). The mixture was stirred at room temperature for 3 hours and poured into 10% HCl and extracted with CH₂Cl₂. The CH₂Cl₂ layer was dried over anhydrous sodium sulfate and concentrated to get the crude product which was further purified by column chromatography over silica gel to obtain compound B0036-6 (400 mg, Yield: 67%, NMR and LC-MS confirmed) as a white solid.

Synthesis of Compound B0036-7

To a solution of compound B0036-6 (400 mg, 1.67 mmol) in anhydrous THF (3.0 mL) at 0° C. was added methylmagnesium bromide (1 M in THF, 3.4 mL, 3.4 mmol). After stirring the reaction at 0° C. for 3 hours, the reaction was quenched with saturated ammonium chloride and the aqueous layer was extracted with CH₂Cl₂. The combined organic layers were dried over anhydrous sodium sulfate and concentrated to get the crude product which was purified by column chromatography on silica gel to obtain compound MED-B0036-7 (212 mg, Yield: 65%, NMR confirmed) as yellow oil.

### Synthesis of Compound B0054

Synthesis of Compound B0053

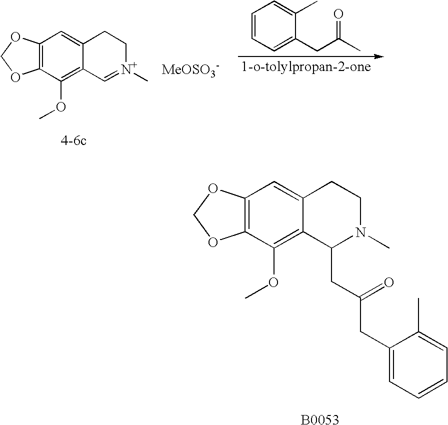

A solution of EtONa was prepared by adding sodium (69 mg, 3.0 mmol) to EtOH (6 mL) at 0° C. and after the pieces of sodium had disappeared, this solution was added dropwise to a solution of compound 4-6c (500 mg, 1.5 mmol) in EtOH (12 mL) at 0° C. and the mixture was stirred for 15 min. 1-o-Tolylpropan-2-one (333 mg, 2.25 mmol) in ethanol (2.0 ml) was added dropwise at 0° C. and the mixture was allowed to stir at room temperature for 2 days. To the reaction mixture was added 1N HCl until the solution reached pH 3-4 and the reaction mixture was concentrated. The acidic solution was washed with Et₂O and the pH was adjusted to pH 11-12 with 1M NaOH. The resulting mixture was extracted with CH₂Cl₂. The CH₂Cl₂ extract was dried over anhydrous Na₂SO₄ and the solution was concentrated under reduced pressure to obtain compound B0053 (500 mg, yield: 90%, NMR and LC-MS confirmed) as a yellow oil.

Synthesis of Compound B0054

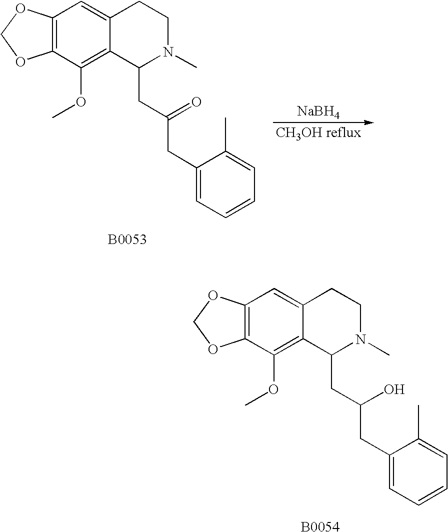

To a solution of 30053 (500 mg, 1.36 mmol) in CH₃OH (30 ml) was added sodium borohydride (103 mg, 2.72 mmol) and the reaction mixture was stirred under reflux for 2 hours. The mixture was cooled and water (5 ml) was added following which the solution was evaporated under vacuum. Water (30 ml) was added to the residue and extracted with chloroform 3 times. The combined organic layers were washed with water and brine. The organic phase was dried with anhydrous sodium sulfate and concentrated under reduced pressure to obtain the crude product (440 mg, yield: 88%, HPLC: 87.6%). The crude product (100 mg) was further purified by preparative TLC to give B0054 (30 mg, NMR confirmed, Purity: 95.4% by HPLC) as a colorless oil.

### Synthesis of Compound B0057

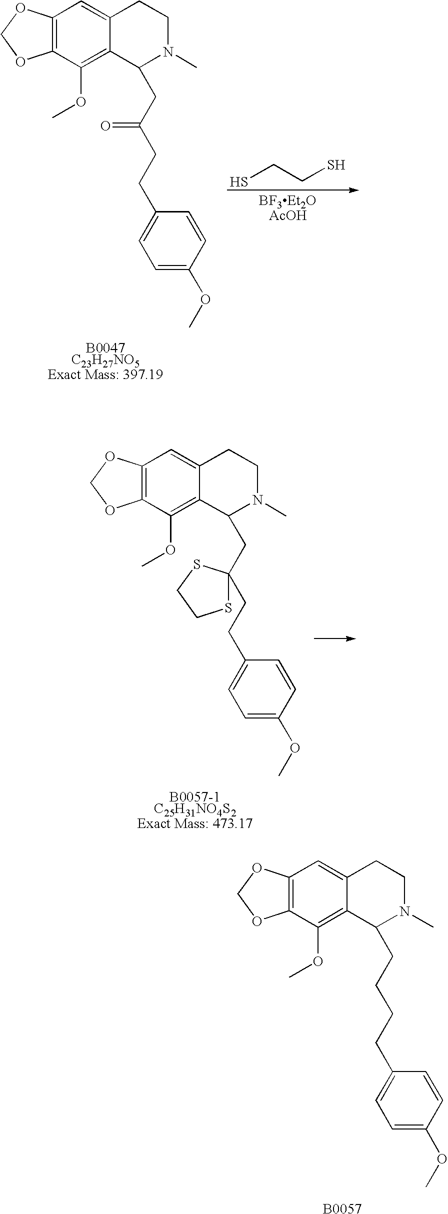

Synthesis of Compound B0047

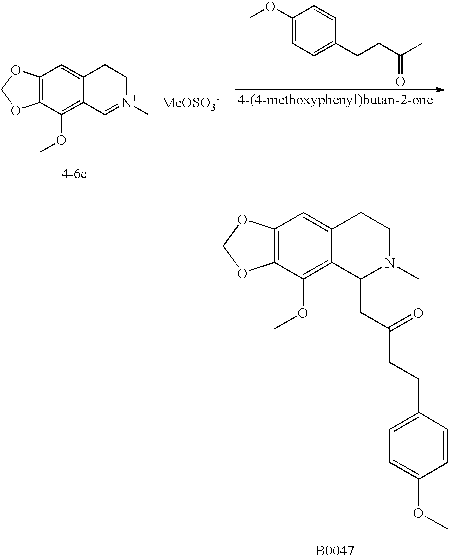

A solution of EtONa was prepared by adding sodium (139 mg, 4.53 mmol) to EtOH (12 mL) at 0° C. under argon atmosphere. After the pieces of sodium had disappeared this solution was added dropwise to a solution of compound 4-6c (1 g, 3.02 mmol) in EtOH (20 mL) at 0° C. and the mixture was stirred for 15 minutes. 4-(4-Methoxyphenyl)butan-2-one (800 mg, 4.53 mmol) in ethanol (4.0 ml) was added dropwise at 0° C. and the mixture was allowed to stir at room temperature for 2 days. To the reaction mixture was added 1N HCl to adjust the pH to 1-2 and the reaction mixture was concentrated. The acidic solution was washed with Et₂O and the pH was adjusted to pH 13˜14 with 1M NaOH. The resulting mixture was extracted with CH₂Cl₂. The CH₂Cl₂ extract was dried over anhydrous Na₂SO₄ and concentrated under reduced pressure to obtain the crude product (1.7 g) as yellow oil. The crude product was dissolved in Et₂O (20 ml) and filtered. The filtrate was evaporated to dryness to obtain B0047 (1 g, Lot#: MC0307-38-1, yield: 83.3%) as a yellow oil.

Synthesis of Compound B0057-1

To a solution of compound 50047 (900 mg, 2.25 mmol) in AcOH (5 mL) was added BF₃.Et₂O (569 mg, 4.5 mmol) and the reaction was stirred for 0.5 hours. Ethanedithiol (639 mg, 6.75 mmol) was added to the reaction mixture and stirred for 24 hours. The solution was poured into H₂O (5 mL) and saturated NaHCO₃ solution was added to adjust the pH to 7-8 followed by addition of NaOH solution to adjust the pH to 13-14. The alkaline solution was extracted with CHCl₃ 3 times. The CHCl₃ layers were combined, dried over anhydrous Na₂SO₄ and evaporated. Further purification by column chromatography (elution with CH₂Cl₂, followed by ethyl acetate) gave B0057-1 (720 mg, yield: 71.8%).

### Synthesis of Compound B0057

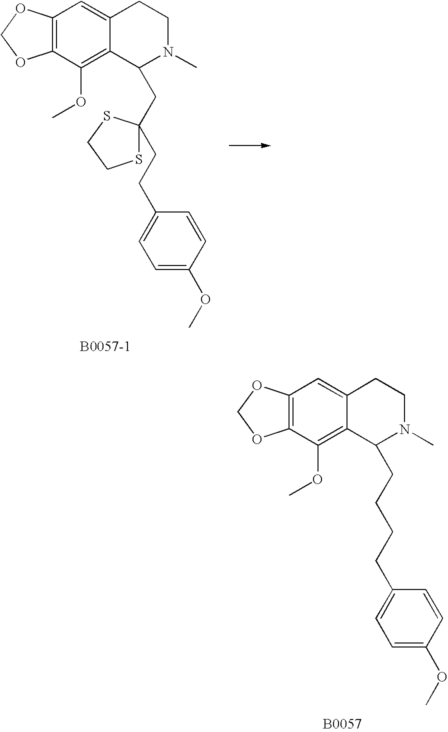

To a solution of compound B0057-1 (400 mg, 0.845 mmol) in ethanol (5 mL) was added Raney Ni (1.5 g) and the reaction mixture was stirred under reflux for 2 hours. LC-MS suggested that part of the starting material remained. More Raney Ni (1.5 g) was added to the solution and the reaction mixture was stirred under reflux for an additional 4 hours. TLC suggested the reaction was complete. The reaction mixture was filtered to remove Raney Ni and concentrated to give the crude product (230 mg, LC-MS confirmed) which was further purified by column chromatography (petroleum ether: CH₂Cl₂=20:1->CH₂Cl₂->CH₂Cl₂:acetone=3:1) to give compound B0057 (120 mg, yield: 31.3%, NMR & LC-MS confirmed).

### Synthesis of Compound MED-B0056

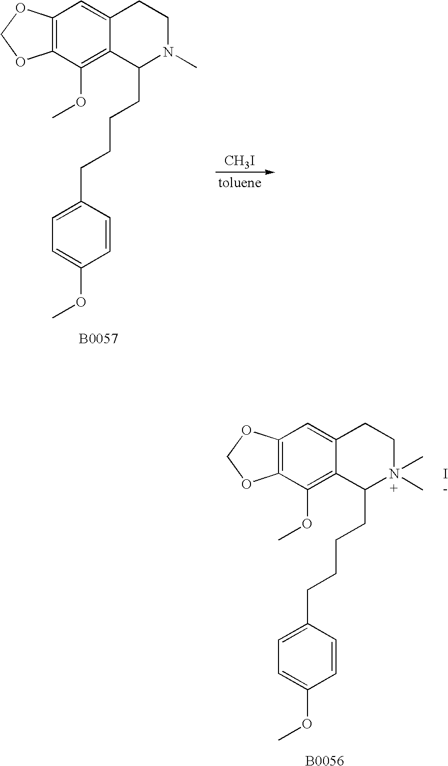

To a solution of compound B0057 (140 mg, 1.05 mmol) in toluene (3 ml) at room temperature was added CH₃I (0.2 ml) and the reaction mixture was stirred overnight (about 18 hours) at room temperature. The yellow oil formed was collected, washed with toluene and dried to obtain the crude product (125 mg, yield: 65%, purity: 92.2% by HPLC). The crude product (65 mg) was washed with CH₃Cl:petroleum ether (1:5) to obtain B0056 (60 mg, NMR and LC-MS confirmed, Purity: 93.3% by HPLC) as yellow oil.

### Synthesis of Compound 3-1

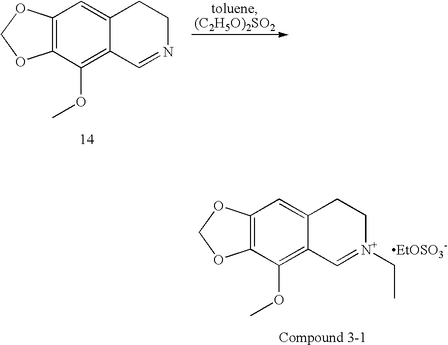

To a solution of compound 14 (4.5 g, 22 mmol) in toluene (200 mL) was added (C₂H_{S}O)₂SO₂ (4.05 g, 26.3 mmol) in portions and the reaction mixture was stirred for 6 hours at room temperature. The reaction mixture was filtered to give compound 3-1 (7.2 g, yield: 92%, NMR and LC-MS confirmed).

### Synthesis of Compound B0072

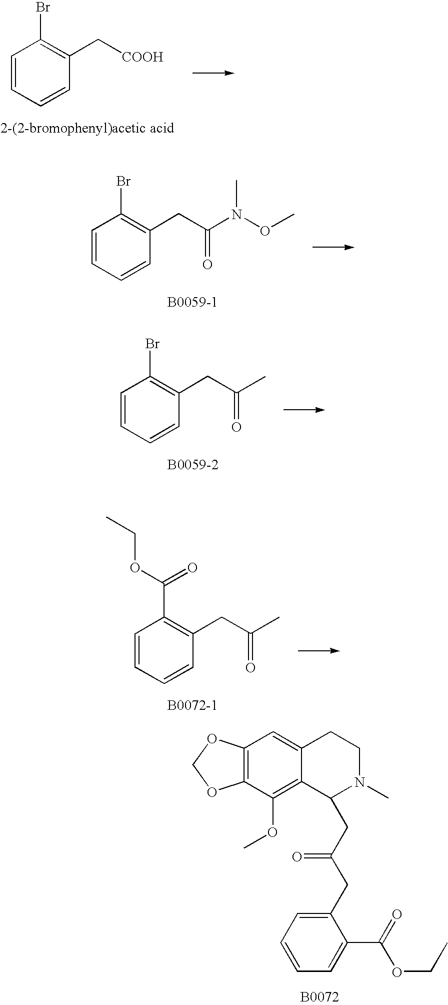

### Synthesis of Compound B0059-1

2-(2-Bromophenyl)acetic acid (5 g, 23.25 mmol) was dissolved in sulfuryl dichloride (11 g, 93 mmol) and stirred at 50° C. for 2 hours following which the sulfuryl dichloride was removed under reduced pressure. The residue was dissolved in CH₂Cl₂ (25 mL) and added to a solution of N,O-dimethylhydroxylamine Hydrochloride (3.4 g, 34.9 mmol) and N-methylmorpholine (5.87 g, 58 mmol) in CH₂Cl₂ (25 ml). The mixture was stirred at room temperature for 2 hours, poured into 10% HCl and extracted with CH₂Cl₂. The organic layer was washed twice with water, four times with 1M NaOH and twice again with water, dried over anhydrous sodium sulfate and concentrated to obtain compound B0059-1 (6 g, crude yield: 99%) as yellow oil.

Preparation of Compound B0059-2

To a solution of B0059-1 (1.8 g, 6.97 mmol) in anhydrous tetrahydrofuran (20 mL) at 0° C. was added methylmagnesium bromide (1 M in THF, 17 mL, 17 mmol) and stirred at 0° C. for 1 hour. The reaction was quenched with saturated ammonium chloride and the aqueous layer was extracted with ether. The combined organic layers were dried over sodium sulfate and concentrated to afford the crude product (1.5 g) which was further purified by column chromatography on silica gel to obtain compound B0059-2 (530 mg, Yield: 35.7%, NMR and LC-MS confirmed) as a light yellow oil.

### Synthesis of Compound B0073

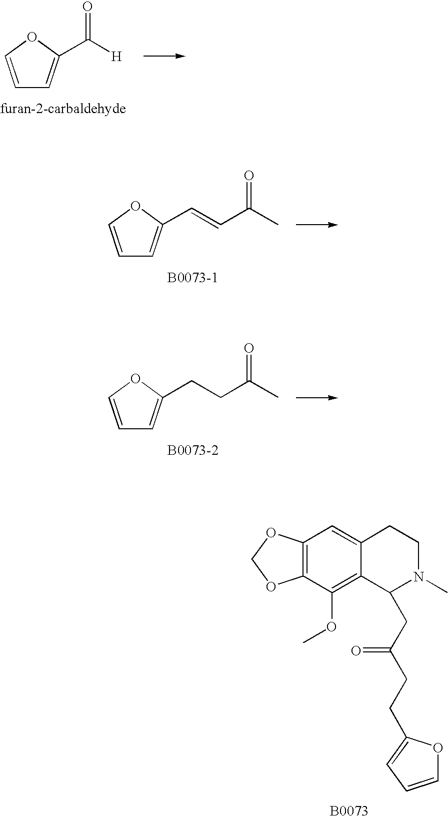

### Preparation of Compound B0073-2

Compound B0073-1 was prepared by standard methods. To a solution of B0073-1 (1 g, 7.4 mmol) in EtOH (25 ml) was added 5% Pd/C (100 mg) and diphenyl sulphide (13.7 mg), and the mixture was stirred overnight (about 18 hours) under H₂ atmosphere at room temperature. The mixture was filtered and concentrated under reduced pressure to obtain the crude product which was purified by column chromatography over silica gel to obtain compound B0073-2 (420 mg, yield: 41.1%, NMR confirmed) as a colourless liquid.

Synthesis of Compound B0073

A solution of EtONa was prepared by adding sodium (93 mg, 4.06 mmol) to EtOH (10 mL) at 0° C. under argon atmosphere. After the pieces of sodium had disappeared this solution was added dropwise to a solution of compound 4-6c (670 mg, 2.03 mmol) in EtOH (13 mL) at 0° C. and the mixture was stirred for 1 hour. Compound B0073-2 (420 mg, 3.04 mmol) in ethanol (2.0 ml) was added dropwise and the mixture was allowed to stir overnight (about 18 hours) at room temperature. To the reaction mixture was added 1N HCl till the solution reached pH 3-4 and the reaction mixture was concentrated. The acidic solution was washed with Et₂O and the pH was adjusted to pH 11-12 with 1M NaOH. The resulting mixture was extracted with CH₂Cl₂. The CH₂Cl₂ layer was dried over Na₂SO₄ and concentrated under reduced pressure to obtain crude product (620 mg) as yellow oil. The crude product was dissolved in ether (5 mL) and the undissolved solids were filtered. Et₂O—HCl was added dropwise to the ether solution till a white solid appeared. This white solid was washed twice with ether, twice with ethyl acetate and dissolved in H₂O (5 mL). The pH of the aqueous layer was adjusted to pH 13-14 with 1N NaOH and extracted with CHCl₃ 3 times. The combined CHCl₃ layers were washed twice with H₂O, dried over anhydrous Na₂SO₄ and concentrated under reduced pressure to give compound B0073 (112 mg, yield: 15.5%, NMR confirmed, purity: 95.4% by HPLC) as a yellow oil.

### Preparation of Compound B0075

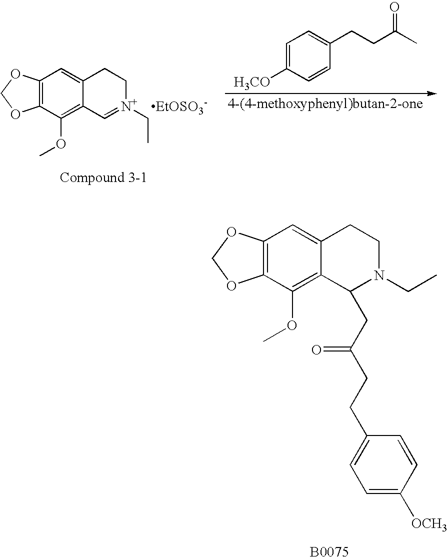

A solution of EtONa was prepared by adding sodium (64 mg) to EtOH (6 mL) at 0° C. and after the pieces of sodium had disappeared, this solution was added dropwise to a solution of compound 3-1 (500 mg, 1.39 mmol) in EtOH (14 mL) at 0° C. and the mixture was stirred for 20 minutes. 4-(4-Methoxyphenyl)butan-2-one (298 mg, 1.67 mmol) was added and the reaction mixture was stirred overnight (about 18 hours) at room temperature.

To the reaction mixture was added 1N HCl (5 mL) and the reaction mixture was concentrated. The acidic solution was washed with Et₂O and the pH was adjusted to pH 13-14 with 1M NaOH and the resulting mixture was extracted with CH₂Cl₂. The CH₂Cl₂ extract was dried over anhydrous Na₂SO₄ and the solution was concentrated under reduced pressure to obtain the crude product (500 mg) as brown oil. This crude product was dissolved in ether (5 mL) and the undissolved solids were filtered. Et₂O—HCl was added to the solution dropwise till a yellow gum appeared. This yellow gum was washed with ether following which ethyl acetate was added to the gum. The gum turned solid. This solid was washed with ethyl acetate, dissolved in H₂O (5 mL), made alkaline to pH 13-14 with 1N NaOH and extracted twice with ether. The combined ether layers were washed twice with H₂O, dried over anhydrous Na₂SO₄ and concentrated under reduced pressure to give compound B0075 (260 mg, yield: 45, NMR and MS confirmed, Purity: 97.5% by HPLC).

### Synthesis of Compound B0076

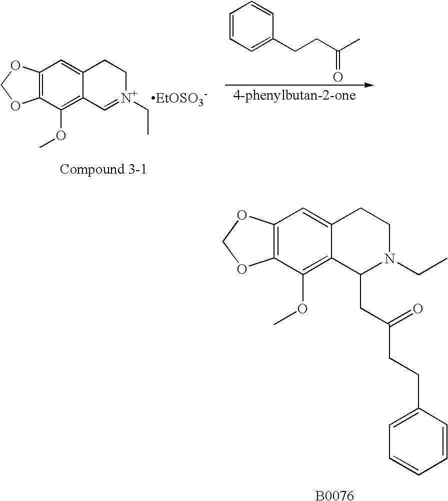

A solution of EtONa was prepared by adding sodium (64 mg) to EtOH (8 mL) at 0° C. and after the pieces of sodium had disappeared, this solution was added dropwise to a solution of compound 3-1 (500 mg, 1.39 mmol) in EtOH (10 mL) at 0° C. and the mixture was stirred for 1 hour. 4-Phenylbutan-2-one (311 mg, 2.09 mmol) in ethanol (2.0 ml) was added dropwise at 0° C. and the reaction mixture was allowed to stir overnight (about 18 hours) at room temperature. To the reaction mixture was added 1N HCl until the solution reached pH 3-4 and the reaction mixture was concentrated. The acidic solution was washed with Et₂O and the pH was adjusted to pH 11-12 with 1M NaOH. The resulting mixture was extracted with CH₂Cl₂. The CH₂Cl₂ layer was dried over anhydrous Na₂SO₄ and concentrated under reduced pressure to obtain the crude product as yellow oil (450 mg). The crude product was dissolved in ether (5 mL) and the undissolved solids were filtered. Et₂O—HCl was added to the solution dropwise till yellow oil appeared. This yellow oil was washed twice with ether and twice with ethyl acetate. The oil turned to a yellow solid which was dissolved in H₂O (5 mL) and the solution made alkaline to pH 13-14 with 1N NaOH. This alkaline solution was extracted with CHCl₃ 3 times. The combined CHCl₃ layers were washed twice with H₂O, dried over anhydrous Na₂SO₄ and concentrated under vacuum to give compound B0076 (218 mg, yield: 41%, NMR and LC-MS confirmed) as yellow oil.

Each of the patents, patent applications and articles cited herein is incorporated by reference. The use of the article “a” or “an” is intended to include one or more.

The foregoing description and the examples are intended as illustrative and are not to be taken as limiting. Still other variations within the spirit and scope of this invention are possible and will readily present themselves to those skilled in the art.

What is claimed:

1. A method of reducing pain in a host mammal in need thereof that comprises administering to that host mammal a pharmaceutical composition containing an effective amount of a compound of Formula I dissolved or dispersed in a physiologically tolerable carrier 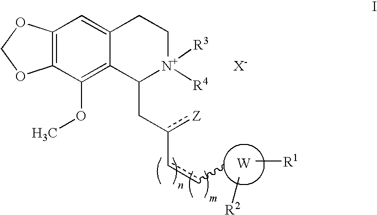
wherein
n=0 or 1;
m=0 or 1;
m+n=0, 1 or 2; 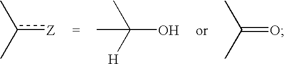
W is a 6-membered aromatic ring containing 0, 1 or 2 nitrogen atoms in the ring;
R¹ is selected from the group consisting of H, C₁-C₆ hydrocarbyl, C₁-C₆ hydrocarboxy, halogen, cyano, C₁-C₆ hydrocarboxyhydrocarboxylene, trifluoromethyl, and hydroxyl;
R² is selected from the group consisting of H, C₁-C₆ hydrocarbyl, C₁-C₆ hydrocarbyloxy, C₁-C₆ hydrocarbyloxyhydrocarboxylene and halogen;
R³ is absent or C₁-C₆ hydrocarbyl;
R⁴ is C₁-C₆ hydrocarbyl;
X⁻=an anion or is absent when R³ is absent;
the dotted line indicates an optional double bond between the depicted carbon atoms; and
the wavy line indicates that the depicted phenyl substituent can be in the Z or E configuration when the optional double bond is present.

2. The method according to claim 1, wherein the sum of m+n is 1 or 2.

3. The method according to claim 1, wherein W contains zero nitrogen atoms.

4. The method according to claim 1, wherein W contains one nitrogen atom.

5. The method according to claim 1, wherein R²═H.

6. The method according to claim 1, wherein R¹ includes an oxygen atom bonded to W.

7. The method according to claim 6, wherein R¹ is C₁-C₆ hydrocarbyloxy.

8. The method according to claim 6, wherein

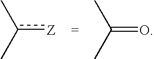

9. The method according to claim 1, wherein R³═CH₃.

10. The method according to claim 1, wherein said compound of Formula I is selected from the group consisting of

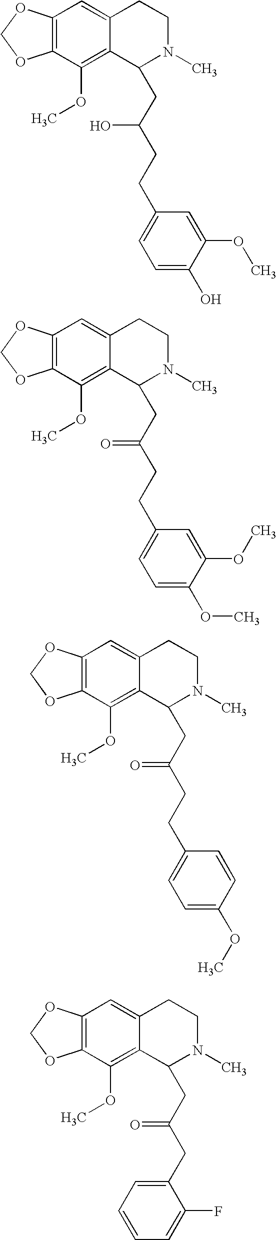

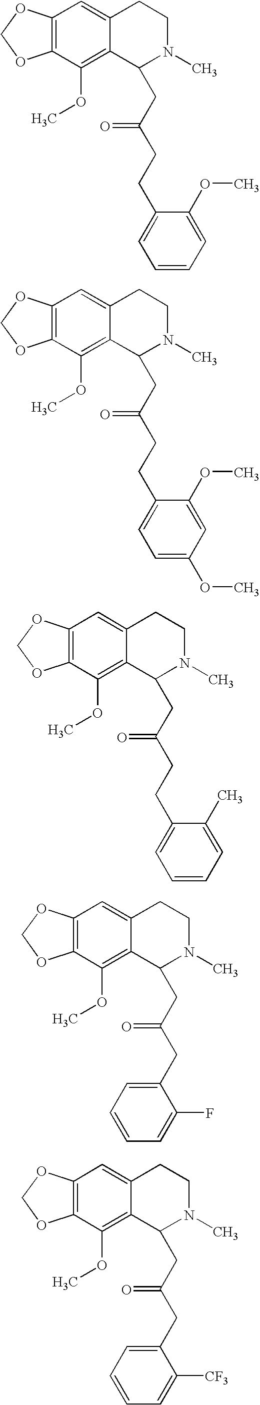

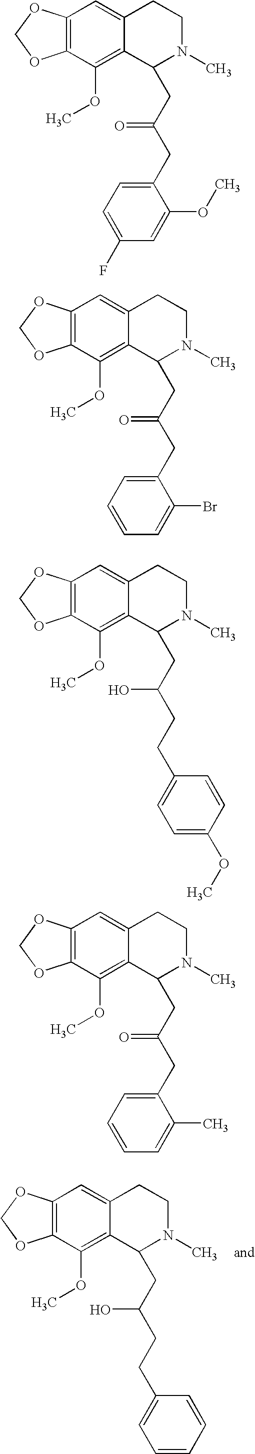

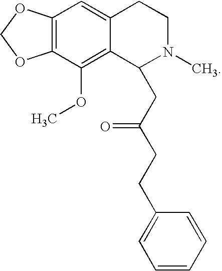

11. The method according to claim 1, wherein said host mammal is selected from the group consisting of a primate, a laboratory rodent, a companion animal, and a food animal.

12. The method according to claim 1, wherein said composition is administered a plurality of times over a period of days.

13. The method according to claim 10, wherein said composition is administered a plurality of times in one day.

14. A method of reducing pain in a host mammal in need thereof that comprises administering to that host mammal a pharmaceutical composition containing an effective amount of a compound of Formula II dissolved or dispersed in a physiologically tolerable carrier 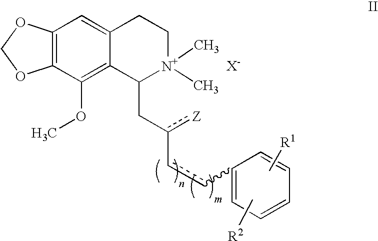
wherein
n=0 or 1;
m=0 or 1;
m+n=0, 1 or 2; 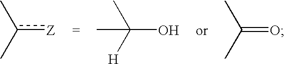
X⁻=an anion;
R¹ is selected from the group consisting of H, C₁-C₆ hydrocarbyl, C₁-C₆ hydrocarbyloxy, halogen, cyano, C₁-C₆ hydrocarbyloxyhydrocarboxylene, trifluoromethyl, and hydroxyl;
R² is selected from the group consisting of H, C₁-C₆ hydrocarbyl, C₁-C₆ hydrocarbyloxy, C₁-C₆ hydrocarbyloxyhydrocarboxylene and halogen;
the dotted line indicates an optional double bond between the depicted carbon atoms; and
the wavy line indicates that the depicted phenyl substituent can be in the Z or E configuration when the optional double bond is present.

15. The method according to claim 14, wherein R²═H.

16. The method according to claim 14, wherein R¹ includes an oxygen atom bonded to the depicted phenyl ring.

17. The method according to claim 16, wherein R¹ is C₁-C₆ hydrocarbyloxy.

18. The method according to claim 14, wherein

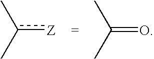

19. The method according to claim 14, wherein said host mammal is selected from the group consisting of a primate, a laboratory rodent, a companion animal, and a food animal.

20. The method according to claim 14, wherein said composition is administered a plurality of times over a period of days.

21. The method according to claim 14, wherein said composition is administered a plurality of times in one day.

22. The method according to claim 14, wherein said compound of Formula II is

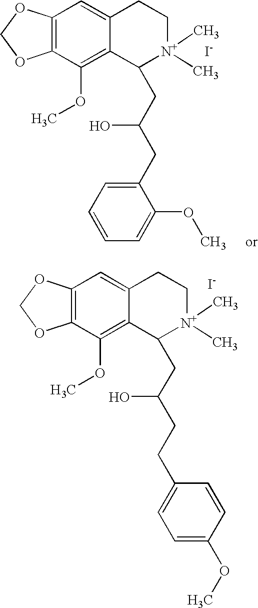

23. A method of reducing pain in a host mammal in need thereof that comprises administering to that host mammal a pharmaceutical composition containing an effective amount of a compound of Formula III dissolved or dispersed in a physiologically tolerable carrier 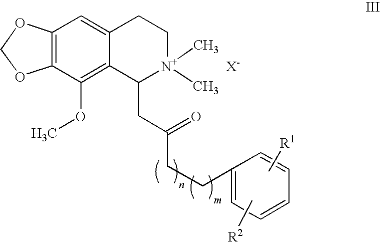
wherein
n=0 or 1;
m=0 or 1;
m+n=0, 1 or 2;
X⁻=an anion;
R¹ is selected from the group consisting of H, C₁-C₆ hydrocarbyl, C₁-C₆ hydrocarbyloxy, halogen, cyano, C₁-C₆ hydrocarbyloxyhydrocarboxylene, trifluoromethyl, and hydroxyl; and
R² is selected from the group consisting of H, C₁-C₆ hydrocarbyl, C₁-C₆ hydrocarbyloxy, C₁-C₆ hydrocarbyloxyhydrocarboxylene and halogen.

24. The method according to claim 23, wherein R²═H.

25. The method according to claim 23, wherein R¹ includes an oxygen atom bonded to the depicted phenyl ring.

26. The method according to claim 25, wherein R¹ is C₁-C₆ hydrocarbyloxy.

27. The method according to claim 23, wherein

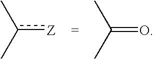

28. The method according to claim 23, wherein said host mammal is selected from the group consisting of a primate, a laboratory rodent, a companion animal, and a food animal.

29. The method according to claim 23, wherein said composition is administered a plurality of times over a period of days.

30. The method according to claim 23, wherein said composition is administered a plurality of times in one day.

31. The method according to claim 23, wherein said compound of Formula III is selected from the group consisting of 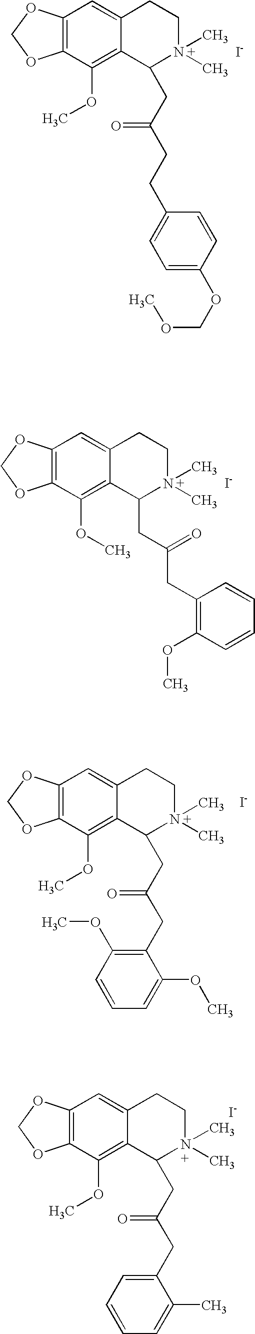 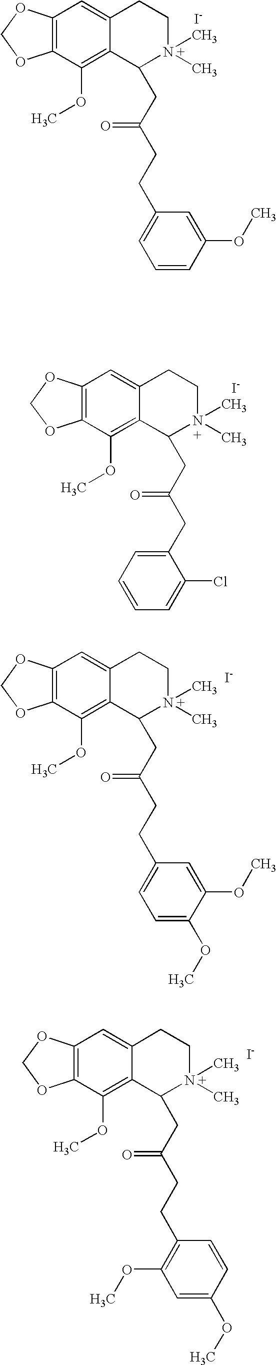 [chemical structure]


SEQUENCE LISTING (1 sequence)

SEQ ID NO: 1 - protein, 5 aa, Artificial sequence
  chemically synthesized FLNA sequence that corresponds to amino acid residue positions 2561-2565 of the FLNA protein
VAKGL